(19) United States
(12) Patent Application Publication
(10) Pub. No.: US 2026/0158392 A1
(43) Pub. Date: Jun. 11, 2026

(54) METHOD AND SYSTEM FOR DEPLOYING STANDALONE AND REACTIVE SQUARES GAMES ADAPTED WITH FANTASY SPORTS AND PREDICTIVE ELEMENTS

(71) Applicant: Frank S. Maggio, Pinellas Park, FL (US)

(72) Inventor: Frank S. Maggio, Pinellas Park, FL (US)

(21) Appl. No.: 19/181,209

(22) Filed: Apr. 16, 2025

Related U.S. Application Data
(63) Continuation of application No. 17/585,281, filed on Jan. 26, 2022, now abandoned.
Application No. 17/585,281 is a continuation-in-part of application No. 16/827,698, filed on Mar. 23, 2020, now abandoned.
Application No. 16/827,698 is a continuation of application No. 15/232,834, filed on Aug. 10, 2016, now Pat. No. 10,596,468.
Application No. 15/232,834 is a continuation-in-part of application No. 14/852,477, filed on Sep. 11, 2015, now Pat. No. 9,858,764.
(60) Provisional application No. 62/049,329, filed on Sep. 11, 2014.
(60) Provisional application No. 62/051,931, filed on Sep. 17, 2014.

(51) Int. Cl. A63F 13/61; A63F 13/20; A63F 13/25; A63F 13/46; A63F 13/65; A63F 13/812; A63F 13/828; C07D 215/46; C07D 401/12; C07D 401/14; G07F 17/32

(52) U.S. Cl. CPC A63F 13/61; A63F 13/20; A63F 13/25; A63F 13/46; A63F 13/65; A63F 13/812; A63F 13/828; C07D 215/46; C07D 401/12; C07D 401/14; G07F 17/3272; G07F 17/3288

(57) ABSTRACT

A system and method are provided for implementing a uniquely automated, and otherwise gamified, version of a Squares Game. The disclosed schemes incorporate technology for hosting on, or interacting/reacting with, potential participants' mobile and personal communicating and computing devices. The disclosed systems and methods uniquely adapt the gamified Squares Game to implement hybrid schemes employed by one or more of the myriad “fantasy sports” leagues that have become attractive to mass audiences. The disclosed schemes provide event-long score tabulation as a mechanism by which to allocate awards. The disclosed schemes provide for branding to certain sponsors of a Super Squares event, league, or season, in a manner that the a sponsoring entity's retail and/or website properties may be linked to the automated game in order to facilitate increased traffic, sales and/or exposure.

### BACKGROUND

This application is a Continuation of U.S. patent application Ser. No. 17/585,281, entitled “Method and System for Deploying Standalone and Reactive Squares Games Adapted With Fantasy Sports And Predictive Elements,” by Frank S. Maggio, filed in the U.S. Patent and Trademark Office (“USPTO”) on Jan. 26, 2022, which in turn is a Continuation-In-Part of U.S. patent application Ser. No. 16/827,698, entitled “Method and System for Deployment of Standalone and Reactive Squares Games Adapted for Employment in a Fantasy Sports League Environment,” by Frank S. Maggio, filed in the USPTO on Mar. 23, 2020, which in turn is a Continuation of U.S. patent application Ser. No. 15/232,834, entitled “Method and System for Deployment of Standalone and Reactive Squares Games Adapted for Employment in a Fantasy Sports League Environment,” by Frank S. Maggio, filed in the USPTO on Aug. 10, 2016, and issued as U.S. Pat. No. 10,596,468 on Mar. 24, 2020, which in turn was a Continuation-in-Part of U.S. patent application Ser. No. 14/852,477, entitled “Methods and Systems for Deployment of Standalone and Reactive Squares Games,” by Frank S. Maggio, filed in the USPTO on Sep. 11, 2015, and issued as U.S. Pat. No. 9,858,764 on Jan. 2, 2018, which in turn claims the benefit of U.S. Provisional Patent Application No. 62/049,329 entitled “Method And System For Deployment Of Standalone And Reactive Sport-Related Game Squares Games,” by Frank S. Maggio, filed in the U.S. Patent and Trademark Office on Sep. 11, 2014, and to U.S. Provisional Patent Application No. 62/051,931 entitled “Method And System For Deployment Variants Of Standalone And Reactive Sport-Related Game Squares Games,” by Frank S. Maggio, filed in the U.S. Patent and Trademark Office on Sep. 17, 2014, the disclosures of all of which are hereby incorporated by reference herein in their entirety.

### FIELD OF THE DISCLOSED EMBODIMENTS

This disclosure relates to systems and methods for uniquely adapting an automated and otherwise gamified Squares Game, which may be hosted on mobile and personal communicating and computing devices, to implement hybrid methods and systems employed by one or more of the myriad “fantasy sports” leagues that have become attractive to mass audiences, and particularly for including additional and predictive/prognosticating elements to participating individual users in the gamified Squares Game to a number of beneficial uses.

### RELATED ART

An extremely popular game played by sports fans, including attendees and viewers of particularly noteworthy football games, including what is commonly referred to as the Big Game, is the “Squares Game.” FIG. 1 schematically illustrates a typical embodiment of a Squares Game matrix form 100. In popular embodiments, a ten by ten square matrix participation area 110 is hand-drawn on paper, posterboard, or the like, or otherwise produced by a host as a game administrator. The host circulates the matrix form 100 among colleagues, associates, friends, co-workers, customers and the like soliciting individual participation in the Squares Game. The matrix form 100 may include certain logos, other identifying information, contact data, instructions or other like inputs in, for example, a free form identifying field 160. The matrix form 100 may contain blank border fields 120,130 outside the ten by ten square matrix participation area 110, e.g., across the top (as shown) or bottom, and down the left side (as shown) or right side, to be filled in when all of the one hundred squares in the matrix participation area 110 have been selected by individual participants.

For the host, the process of soliciting participation in filling in the matrix participation area 110 by “selling” the squares, one square or a couple of squares at a time, can be a time-consuming and frustrating undertaking as enthusiasm for participation among a narrow population of participants with whom the host may be associated and/or interact ebbs and flows. Some of this burden on the host may be eased by “selling” the individual squares in pre-planned groups of four (as is indicated in FIG. 1 by the slightly heavier lines in the matrix participation area 110) as, for example, “Super” Squares.

After selling (or otherwise simply administratively filling) all of the squares in the matrix participation area 110, the still blank border fields 120,130, may be filled in with “score heading” numbers in the manner shown in FIG. 1. These score heading numbers, in the context of a conventional football pool, corresponding to the final digit of each of the “Home” and “Away” (see elements 140,150) team scores for the game, or alternatively for selected intervals throughout the game, such as, for example, by halves or by quarters, according to a pre-determined and pre-announced prize structure. Although cumbersome, in instances in which awards may be assigned with each change in score, the score heading numbers may be separately randomized, redistributed and/or reset each time the score changes. The score heading numbers entered into the border fields 120,130 are typically randomly selected one at a time, and placed at the top or left of a column or row in the example shown in FIG. 1, respectively.

The letters A-Z (minus Q) are described in view of the disclosed embodiments below.

Participants in conventional paper Squares Games may, for example, may pay, for example, $5 per square, for a total prize pool of $500. The collected sales proceeds from the conventional paper Squares Game may then be divided among the participating individuals, typically based upon the score of the game, or at the pre-determined intervals throughout the game, or at every change of score. As a common example in the conventional paper Squares Game, the score at the end of each quarter of a football game may garner the “winning” participating individual a 25% increment of the overall prize pool, or these scores may garner 20% increments for the first 3 quarters, and 40% for the end of game score. In some instances, the host, administrator or organizer of the conventional paper Squares Game may retain a portion of the square sales proceeds, to offset a “cost” of managing or administering the Squares Game. These types of conventional paper Squares Games are generally informally administered among groups of friends and/or colleagues, and the “rules” are generally pre-briefed to all participating individuals who then voluntarily agree to participate according to those rules.

There are many variations on this general theme and in establishment of the matrix participation area 110 as shown in the exemplary embodiment illustrated in FIG. 1, all of which may be pre-established and explained to participating individuals. Further, although the concept of a Squares Game is generally associated with football games, the concept of the conventional paper Squares Game may be adjusted and adaptable to other sports, in many unique ways.

### SUMMARY OF THE DISCLOSED EMBODIMENTS

With the proliferation of individual mobile communicating and computing devices, an opportunity exists to substantially automate a conventional paper Squares Game in a manner that increases user participation and interest, and to incorporate participation in such games on a local, regional or broader scope into an overall reactive advertising scheme in which participating individual users may be awarded and/or rewarded, not only for the users'participation in a particular game, but more broadly for the users'participation in a reactive advertising environment overall.

In view of the above background, it may be advantageous to implement a scheme by which to automate a conventional paper Squares Game in a manner that achieves the above advanced objectives. In this regard, it may be particularly advantageous to provide programming for participating individual users'mobile communicating and computing (“smart”) devices that may facilitate the employment of those smart devices to awarding, rewarding, challenging and interesting automated gamification of, and reactive advertising associated with, a Squares Game. An objective may be to employ the automated Squares Game as an entry into a reactive advertising scheme thereby generating, or otherwise stimulating, a new degree of interest in integrated commercial and advertising content.

Moreover, as the size of the population of participating individual users increases, it may be appropriate to incorporate certain additional incentives, and/or to separately incorporate certain additional discriminators to fully “qualify” a particular participating individual user as an interval or overall game “winner.”

Exemplary embodiments of the systems and methods according to this disclosure may provide unique automation of a Squares Game for participation across a broader spectrum of a participating individual user population.

Exemplary embodiments may implement automated schemes directed at overcoming the burden of hosting a local Squares Game in a limited participant population environment.

Exemplary embodiments may ease the logistical burden on any host, administrator or organizer of a Squares Game in at least one of automating (a) the selecting, assigning or “selling” of the squares in the matrix, (b) the selecting or assigning of the numbers associated with the squares matrix for the game, at intervals of the game, or according to virtually any randomized scheme throughout the game, and (c) the preparing of the finalized squares matrices to be automatically distributed to all participating individual users prior to an event to which the Squares Game is linked.

Exemplary embodiments may distribute participation across a broader user population than simply the potential participating individual users, e.g., 100 or so people, that the host, administrator or organizer knows.

Exemplary embodiments may verify receipt of the finalized squares matrices with all of the participating individual users in order to ensure that attention remains high.

It is recognized that some participating individual users'interest in, for example, the individual or multiple sporting events with which a Squares Game may be associated is often specifically increased based on the vested interest of those some of the participating individual users in the outcome of each prize distributing interval across the individual or multiple sporting events with which the Squares Game may be associated. Part of the allure of the concept of the conventional paper Squares Game is that it adds excitement to sporting events, particularly when a favored team is not winning, or when the game is no longer close.

Exemplary embodiments may incorporate additional elements that are divorced from the individual or multiple sporting events with which the disclosed Squares Game is associated in order to maintain the attention of the population of participating individual users regardless of the “outcome” of the individual or multiple sporting events.

Exemplary embodiments may incorporate in-game predictive, reactive, quizzing and matching elements individually, or in myriad combinations, to maintain the attention of the participating individual users throughout the game.

Exemplary embodiments may employ incorporated in-game predictive, reactive, quizzing and matching elements individually, or in myriad combinations, particularly as the population of participating individual users increases to discriminate individual winners among, for example, a large number of participating individual users all of whom have a particular matching square throughout the game.

Exemplary embodiments may specify a hierarchy of individual elements and actions with regard to the incorporated in-game predictive, reactive, quizzing and matching elements individually, or in myriad combinations, that must be undertaken by participating individual users to qualify for certain prizes or prize pools, for example.

In embodiments, myriad combinations of predictions, reactive quizzes, and/or square matching elements may be considered to provide a robust description of a game of skill that would qualify such an augmented Squares Game, (for example a “Predicter Squares Game”) for wagering deployment via sports books and casinos.

Exemplary embodiments may further incorporate additional fantasy sports elements, such as, for example, the selection or drafting of players or squads for a mock or fantasy team, as a game of skill that is currently permitted in all fifty United States, to avoid legal or jurisdictional encumbrances that may be associated with requirements for gaming licensure that is consistent with, for example, prop bets or parlays. Where appropriate, or “required,” exemplary embodiments may include those “fantasy” elements appropriate to ensure compliance with local statutes, regulations and/or standards.

In embodiments, by supplementing or replacing certain predictive elements of a predictive element enhanced Squares Game, with certain fantasy sports elements including selection of players or team units, the addition of score-matching squares and reactive quiz gamification is intended to ensure that such augmented Squares Games according to this disclosure fall within the scope of a fantasy wagering game. A resulting combination would also provide a unique and exciting new game of skill that many in the population of participating users may additionally enjoy.

Exemplary embodiments may provide automated notifications to winners from among the population of participating individual users in a particular Squares Games once those winners are selected according to squares matching, and where appropriate, application of a specified hierarchy of additional discriminating elements as outlined above.

Exemplary embodiments may execute an automated randomization scheme for selecting the numbers to fill the border areas in a manner that reduces any possibility for controversy with the host based on the ultimately selected numbering scheme.

Exemplary embodiments may execute the automated randomization scheme for selecting the numbers to fill the border areas at various specified intervals throughout the duration of the Squares Game according to specified triggering events, including preplanned time intervals in a sporting event, or score changes in the sporting event.

Exemplary embodiments may collect participant information for recurring gaming or to separately solicit participation in reactive advertising that may be of interest to certain of the participating individual users.

Exemplary embodiments may implement hybrid methods and systems used by myriad “fantasy sports” leagues by providing, for example, event-long score tabulation, and/or player/team drafting, as other mechanisms by which to allocate awards.

Exemplary embodiments may provide for branding to certain sponsors of a Super Squares event, league, or season, in a manner that the a sponsoring entity's retail and/or website properties may be linked to the automated game in order to facilitate increased traffic, sales and/or exposure. In embodiments, an involvement of sponsoring entities may contribute to increased funding for prizing, which may result, in turn, in increased attraction of participating individual users leading to increasing revenues realized by a hosting entity and/or related parties.

These and other features, and advantages, of the disclosed systems and methods are described in, or apparent from, the following detailed description of various exemplary embodiments.

### BRIEF DESCRIPTION OF THE DRAWINGS

Various exemplary embodiments of the systems and methods for uniquely adapting an automated and otherwise gamified Squares Game, which may be hosted on mobile and personal communicating and computing devices, to implement hybrid methods and systems employed by one or more of the myriad “fantasy sports” leagues that have become attractive to mass audiences, with application across many sporting events and sports types, including predictive and reactive quiz elements, according to this disclosure, will be described, in detail, with reference to the following drawings, in which:

FIG. 1 schematically illustrates a typical embodiment of a Squares Game matrix form that may be automated according the disclosed schemes;

FIG. 2 illustrates a block diagram of an exemplary system for implementing an advanced automated Squares Games according to any of the varied schemes outlined in detail below in this disclosure;

FIG. 3 illustrates a flowchart of a first exemplary method for implementing an advanced automated Squares Game according to this disclosure; and

FIG. 4 illustrates a flowchart of a second exemplary method for implementing an advanced automated Squares Game according to this disclosure.

### DETAILED DESCRIPTION OF EMBODIMENTS

The systems and methods for uniquely adapting an automated and otherwise gamified Squares Game, which may be hosted on mobile and personal communicating and computing devices, to implement hybrid methods and systems employed by one or more of the myriad “fantasy sports” leagues that have become attractive to mass audiences across many sporting events and sports types, according to this disclosure will generally refer to this specific utility for those systems and methods.

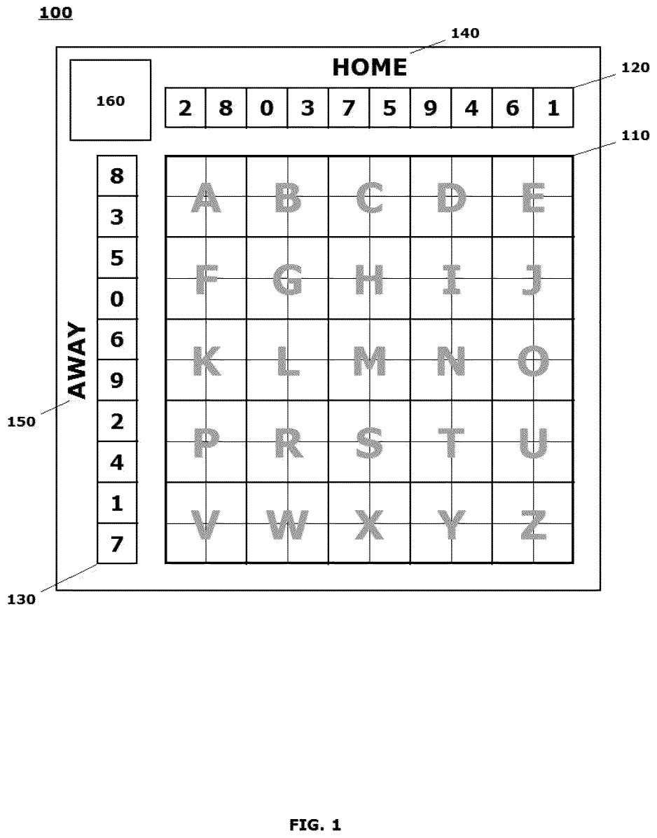

Exemplary embodiments described and depicted in this disclosure should not be interpreted as being specifically limited to any particular physical configuration of a user mobile computing or communicating device, or to any particular class of, or protocol for, networked communication among devices of participating individual users. In fact, the disclosed systems and methods are not necessarily even restricted to user mobile devices. An electronic display component, which may display, for example, a matrix such as that shown in FIG. 1 may be usable to implement the disclosed schemes. Such display screens may include those associated with, for example, an Internet-based implementation accessed through a host website.

It should be recognized that any advantageous use of the systems and methods for gamifying a Squares Game, and for providing the gamified Squares Games to a broad cross-section of a population of participating individual users, in a manner that not only promotes participation in the games, but also provides for information exchange pursuant to immersion of a user in a reactive advertising scheme or environment that may benefit from processes, techniques or schemes such as those discussed in detail in this disclosure is contemplated as being included within the scope of the disclosed exemplary systems and methods.

The systems and methods according to this disclosure will be described as being particularly adaptable to enhancing a gamification experience related to randomized participation in a Squares Game related to one or more sporting events, and adaptations of such gamification as may incorporate certain features that have emerged as being particularly attractive in the context of fantasy sports leagues, and fantasy sports gaming.

Among these features are those that may be adapted from the emergence of fantasy sports wagering, which typically comprises a variety of gaming methods by which participating individual users may select from among one or more team units or players, (such as “TEAM Defense” or “Sammy STAR, QB,”) to compile a “fantasy team” that may aggregate certain accumulated points over the course of a football game, or a period of multiple football games. Points are typically awarded based upon the ability of the specified units or players accomplishing myriad activities during the sporting event(s). Activities that may result in points being collected by, or allocated to, the accounts of particular participating individual users following a football game or games may include pass receptions, runs, aggregate passing yards, touchdowns, sacks, turnovers caused/recovered and the like.

In embodiments, players may be, for example, selected to fill a defined number of positions, which may ultimately include a number of “fantasy” players that simulate a real-world line up (or “roster”) of players and positions. In embodiments, fantasy rosters may require only that a pre-determined number of players and positions be filled, selected from a pool of eligible choices. A particular game may require, for example, a participating individual user to select one quarterback, two running backs, two wide receivers, and a number of players to fill a defensive unit, where the possibility exists that multiple participating individual users may eventually select similar, or even identical, rosters.

In embodiments, fantasy sports games may allocate a fantasy “salary” to each player, where a superior player projected to earn more fantasy points may require more salary to be selected or drafted. By providing participating individual users with a salary cap from which players may be selected, the most favored players may not be eligible for selection before the salary cap is surpassed. Therefore, salary cap management enhances the skill component of fantasy games, as participating individual users may need to evaluate a projected cost-benefit value of each drafted player.

In embodiment of fantasy sports games, players available for drafting may become available and bid upon from salary caps in an auction, snake-draft, or other ordering method such that once selected, no other participating individual user may be afforded the opportunity to select the player. This may sometimes be referred to a Draft Fantasy team.

Those familiar with versions of these gaming features may be aware of various other scoring and draft game mechanics deployed by hosts, administrators or organizers of fantasy games, that may be applied to a Fantasy or Draft Squares game.

In embodiments, by combining the fantasy and draft elements into a sports Squares Game, or other stat-matching type of game, participating individual users may be afforded an opportunity to enjoy the combination of fantasy and/or draft elements, including salary cap and position-based player or team unit selection, with the score or stat-matching elements of games such as Super Squares, and Super Squares supplemented with predictive elements (which may be referred to as Predicter Squares.

In embodiments, the disclosed systems and methods may have the advantage of collecting demographic (and even personal) information regarding participating individual users in a manner that provides advertising, marketing and/or sponsoring entities with a body of analyzable data regarding those participating individual users in order to target advertising content, including reactive advertising content, at those participating individual users. In this regard, an amount of Business Intelligence regarding a population of participating individual users may be collected and made available, or otherwise provided for use, to interested parties and participating entities.

Specific references to gamification and information exchange schemes are meant to be illustrative only in providing examples of real-world utility for the disclosed systems and methods, and should not be considered as limiting the disclosed systems and methods to any particular product or combination of devices, or to any particular type of electronically or physically sharable data vehicle. In other words, any commonly-known user personal electronic, computing, communicating and/or data display component, whether substantially fixed, or easily mobile, and including myriad emerging wearable technologies, may be incorporated into the overall scheme for automated gamification of the Squares Game.

In addition to the above-noted shortfalls in conventional paper Squares Games, those that have participated will recognize that certain score combinations (such as, for example, 2,2 or 5,2 in football games) are extremely infrequent winners of Squares Games. Historically, quarters and entire games of football infrequently end in scores that reflect these score combinations. Therefore, in conventional paper Squares Games, holders of unfavorable score combinations receive little of the additional entertainment value, from the very beginning. The disclosed schemes may incorporate certain weighting, or in process randomization, schemes to be applied once the random numbers are chosen to better “level the playing field” in a manner that enhances participation for all participating individual users even in the context of a single sporting event. For example, according to a pre-determined and pre-briefed schemes, periodically throughout the sporting event with which the Squares Game is associated, random number combinations may be shuffled, or in embodiments, separately selected for the reward of additional “prizes.”

The disclosed schemes are intended to take Squares Games, which are infrequently hosted outside of the Big Game (one of the most widely viewed sporting events in the world every year), or are otherwise infrequently hosted by recurring basis due to their manually cumbersome nature, and provide a level of automation that may foster broader adoption of Squares Games over the balance of football games, and indeed across a more significant cross-section of other professional sports. A capacity of the disclosed schemes to reach a broader participating population may render the schemes more popular in their ability to attract many people without being tied, for example, to the restrictions of a closed viewing area among a controlled group in which a conventional host, administrator or organizer can most easily manually conduct, manage, play and/or oversee the Squares Game.

Moreover, as the population of participating individual users increases, so does the potential for multiple participating individual users to end up with a same matching square. In embodiments, additional “fantasy,” “predictive,” “reactive quiz,” and other elements, and combinations thereof, may be presented to provide additional discriminators or distinguishing elements by which to select and/or identify ultimate game winners.

As will be described in greater detail below, the variability of the structure of the game application, including in-event re-randomization of the matrix, and the addition of user interactive elements and features, may promote higher levels of participation, and continued interest for particular participating individual users during an entirety of the sporting event with which the Squares Game is associated.

The disclosed schemes may prove particularly advantageous for many popular places where fan groups congregate and consume sports (and food and beverages-potential advertisers for Squares Games), but which traditionally do not lend themselves easily to implementation of a conventional paper Squares Game, because the participating environment does not lend itself to adequate oversight of game play. Additionally, the disclosed schemes may prove particularly beneficial as additional incentives or “perks” at professional and collegiate sports venues in addition to sports bars, and, virtually any venue that may attract large viewing, including television viewing, audiences. In implementations, it is conceivable that tens of thousands of fans may participate in a plurality of individual device-implemented Squares Games that are not tied to any particular venue. Viewership and participation may be beneficially increased as populations of participating individual users may be naturally inclined and interested to play.

The disclosed schemes may provide an easy and controlled way to educate, aggregate and enable large numbers of simultaneous players, even on multiple Squares Games among a particular group of participating individual users. In this regard, the disclosed schemes are intended to render the conventional paper Squares Games, and innovations thereof, substantially more ubiquitous. For example, it is not inconceivable that a million or more participating individual users may be accommodated to simultaneously participate in a single aggregated game related to a particular sporting event, or set of sporting events, with prizes and awards being allocated according to a predetermined scheme that makes a Grand Prize very attractive to the large population of participating individual users, and yet is capable of rewarding different levels of awards and prizes to a broad spectrum of participating individual users according to a scheme of participation that includes several levels of, for example, tiebreakers.

In embodiments, variations on the conventional scheme are proposed that provide an increasingly robust, sophisticated and engaging employment of a basic automated Super Squares technology. Among other objectives, these increasingly sophisticated schemes may serve to increase the likelihood of mass adoption of the Super Squares methods and systems, and adaptations to myriad associated real-world sporting events by implementing hybrid methods and systems adapted from the types of interactions that have emerged for use by fantasy sports leagues, which are becoming increasingly attractive to tens of millions of participants.

In embodiments, fantasy or draft features may replace, or more likely supplement, prediction elements with the addition of one or more fantasy player or unit choices.

In embodiments, participating individual users may be allocated a salary cap of X (sixteen (16), in this example) “salary units,” and may be required to select five (5) players from all players participating in a professional football game that week. The “roster” of those five players to be “drafted” may include, for example: 1 Quarterback, 1 Running Back, 1 Wide Receiver or Tight End, 1 Kicker, and 1 Defensive Unit. By way of one more detailed example, assume that a maximum “salary” assigned to the best projected players in each position category is five (5) units, and the least is one (1) unit, with all eligible active players being priced between these extremes (perhaps even to decimal places, such as, for example 3.75 points for a particular quarterback). In embodiments, some combination of predicters may be provided, or three of the predicters may be replaced by three players or team units.

In embodiments, participating individual users may select the roster, after which the participating individual users may be assigned a first random single-combination square or a four-combination Super Square. Alternatively, in embodiments, participating individual users may select and purchase the first square or Super Square. Subsequent squares or Super Squares may be otherwise assigned or purchased pursuant to the rules of the game being played.

In embodiments, an objective of adding discriminators from among the elements described above is to reduce, or substantially prevent, the likelihood of a tie as may emerge with a large population of participating individual users being assigned squares, or “super” squares, in a 10×10 matrix. In embodiments, each participating individual user may answer one or more predictive questions, such as, for example, “Predict the final score of the Monday Night Football Game”. Any predicters may be evaluated or “scored” based on a formula or algorithm that would provide a higher score based on proximity of each prediction to the final outcome.

In embodiments, a score that is modified based on a prediction may only be used when a tie between participating individual users occurs. In embodiments, the one or more predictions may always be included in the final score calculation for all participating individual users. In an event the prediction element is always included to arrive at a final score, the maximum score value for perfect predictions may be nominal, equally weighted, or of a higher priority when compared to the points associated with fantasy or draft points, and square matching points.

In embodiments, participating individual users may be afforded an opportunity to “slide,” jump, or otherwise alter the square or Super Square combinations assigned or selected at the commencement of each event “round” or “quarter.” By way of illustration, a “slide” would allow participating individual users to move their square or Super Square to an adjacent square moving along the “X” or “Y” axis, or it may “jump” to an adjacent square at a diagonal to a participating individual user's square or Super Square. The effect of a slide would allow a participating individual user to alter the digit associated with one, but not both, of the scores associated with the initial square or Super Square. The effect of a jump would allow a participating individual user to alter the digit associated with both of the scores associated with the initial square or Super Square. Using a King on a chessboard, a slide would equate to a move forward, backward, left, or right, while a jump would equate to a diagonal move upward left or right, or backward left or right. Another type of move may be a “hyper jump,” which would enable the square or Super Square to be relocated to another randomly selected square or Super Square. In embodiments, participating individuals might relocate their square or Super Square to the location of their choice. In such instances, participating individual users may be required to use saved salary cap units (instead of, or in addition to making a wager or additional cash payment) in order to be eligible to alter the square locations. By way of example, as an incentive to “save salary” cap, for each salary unit NOT invested in a player salary, a participating individual user may be allowed a single square “slide” per quarter (or sporting event).

In embodiments, should a particular participating individual user be permitted a salary cap budget of 20 units, but require 1 salary cap unit to slide a Super Square once each quarter, by spending 16 or fewer points on player salary, the saved 4+ units would permit the particular participating individual user to make 4 slides over the course of the event.

In embodiments, saved salary cap units may be applied as a discount to the cost of the square slide. For example, the cost to make a square slide may be $5, but a particular participating individual user having saved 3 salary units, may be permitted to exchange one or more of the save salary units for dollars, thereby (according to this example) being afforded an opportunity to pay only $2 to move/slide a square. Alternatively, any such “discount” may, in embodiments, be applied to ALL square slides for the balance of a particular game.

In embodiments, unspent or saved salary cap units may form a multiplier or bonus for the total score achieved for a Squares Game. For example, should a particular participating individual user save 2.3 salary cap units of 20 salary cap units available for a particular game, the 2.3 saved salary cap units may be applied as a multiplier to one or more of the scores achieved by drafted players or team units, or as a multiplier for one or more squares or Super Squares matched over the duration of the game.

By way of example, the rules may permit the saved salary cap units to be multiplied against the total fantasy points earned by the defensive unit, such that in the event a drafted defensive unit scores 12 fantasy points, the 12 points would be multiplied by 2.3, to arrive at an adjusted fantasy point score for the defensive unit of 27.6. In embodiments in which saved salary cap units are to be applied as a multiplier for the score associated with the square or Super Square, points earned from the fourth and final Super Square, for example, where a Perfect Super Square (defined as a Super Square containing (matching) the correct score for both teams) would normally earn 7 points, it would instead earn 7×2.3 or 16.1 points.

In embodiments, unspent or saved salary cap units may simply be added to the final overall score for, for example, a fantasy squares wager, or might be used even as a tie-breaker in the event two or more participating individual users are tied.

Further objectives of the disclosed schemes may include providing elevated branding to certain entities such as marketers, advertisers and like forms of traditional sponsors that may desire to become involved locally, regionally or nationally in one or more Super Squares events, leagues, seasons or the like, in a manner that the “sponsors'” retail and/or website properties experience increased traffic (and therefore sales and exposure). It is anticipated that brands may be willing to subsidize or increase marketing spend for participation in one or more Super Squares schemes with an advantage of increasing funding for prizing, which can in turn increase the population of participating individual user (and revenues) to the host and related parties.

The disclosed schemes may implement the popular Squares Game to achieve the above objectives. Embodiments may provide an automated opportunity to invite participation in simultaneous Squares Games associated with one or more sporting events, which also may be occurring actually or nearly simultaneously. Embodiments separately may provide an opportunity for a particular group to all participate in a same Squares Game in a manner that maintains the familiarity of playing the game among a group of friends, associates, colleagues, co-workers or the like. The disclosed schemes may provide an additional advantage that, on occasions where a number of participating individual users in a particular group may not be enough to “fill” a particular matrix, other participating individual users may be “allowed” into the particular game to maintain the fidelity of the game, the odds of any particular participating individual user winning and the prize pool at requisite levels.

Based on the automated nature of the disclosed embodiments, these schemes may have particular other advantages over manual implementations in that, in embodiments, score combinations may be changeable among specified game periods within the Squares Game in a manner that provides heightened excitement to all, by enabling participants to avoid being left with unattractive score combinations for an entire duration of a particular sporting event.

In embodiments, limitations that may be associated with conventional paper Squares Games may be substantially eliminated through automation according to the disclosed schemes.

In embodiments, for example, an event-long score tabulation and/or some manner of “Leaderboard Overlay” may be provided. Squares Games may provide an opportunity for what could be considered “games within a game.” Generally, each “round” (typically tied to a particularly measurable duration or occurrence within the associated lives sporting event) provides a chance to win a prize, and each new round (new quarter, new inning, new period and the like) is its own self-contained event. Participating individual users without a matching score, according to conventional schemes, essentially win nothing. In embodiments according to the disclosed schemes, it is possible to make participating individual user participation more attractive by modeling the Squares Game experience to mirror certain differing (and apparently successful) elements of “Fantasy Football” gaming. In embodiments, a leaderboard and/or scoreboard may be provided to reflect an ongoing (protracted or multiple-event) competition, or tournament-type, scheme between individual player participants, groups of player participants, or a virtual national audience.

In embodiments, the disclosed schemes provide a capacity to track and tabulate large volumes of participating individual user data in order that, for example, a tournament-type competition may be automated in a manner that would allow it to transpire across a full length of a sporting event broadcast (even involving certain specified events that may or may not occur in a pre-game and/or post-game section of a broadcast (televised) event, or a live venue event). In embodiments, scores may be tabulated and updated for each “round,” and leaderboards may be provided to participating individual users on their individual mobile devices to allow the participating individual users to determine and track individual overall standings against an entire population of participating individual users “competing” at any given time.

The disclosed schemes may provide flexibility to determine and assign participating individual users to participation against subsets of an entire population of participating individual users as well. In embodiments, a particular leaderboard may be established, for example, to serve and/or support a localized (private or public) tournament involving some subset of all participating individual users. In this manner, “private” leagues, or even friends who are hosting, for example, a Big Game party, or associated with some other viewing type event, may be specified. Regardless, however, advertising content associated with the event, the host, a sponsoring entity, or the like may be provided in order that a prize pool may reach beyond the contributions of a small number of participating individual users. In embodiments, a series of local leaderboards representing sub-groups within a section of a stadium, sports bar, sports book or other participant-gathering venue may be specified.

In embodiments, a “national” or “global” leaderboard may be derived and implemented from an aggregate of some plurality, including all, local groups associated with a particular event. Such a broad-based leaderboard may provide all participating individual users with standings as to how individual results compare among the population of all other participating individual users in the aggregate. Such a national leaderboard may aggregate a large enough population of participating individual users, and/or may be more attractively sponsor-entity funded and supported, in a manner that supports the awarding of comparatively larger numbers of more significant prizes, when compared with local groups that may include only enough participation to support the award smaller prizes, or merely to provide individual ones of the population of participating individual users with local bragging rights. Additionally, or separately, winning a local leaderboard may also provide a local winner an opportunity to access another (broader) prize pool. In embodiments, a participating individual user designated as a local winner may be afforded an opportunity to participate in some manner of playoff or tie-breaker round, by gaining access to other “selected” tournaments/events, or by being entered in a separate “super” or “grand” prize drawing, or otherwise may be awarded additional “perks.”

In embodiments, for example, an event-long tournament and leaderboard may take a form of awarding points for various achievements, or levels of achievement, accomplished by participating individual users in each round, or according to any specified periodicity, accruing these points, for example, over a course of a sporting event, and displaying the accrued points via one or more separate leaderboard displays. Fields may be provided for a showing of fantasy, predictive, reactive quiz and square matching “points” associated with each category. In embodiments, participating individual users may, for example, accrue points (or other markers, prizes, credits, or devices that can be collected and measured) for (1) performance of the users'fantasy selections, (2) the users'predictions of certain outcomes, (3) the users'responses or answers to reactive queries or questions, in addition to a tracking of the users'squares matching performance. The reactive queries or questions may include inquiries regarding details of product placement in an event broadcast, advertising content presented in an advertising insert provided on the participating individual users'displays (including according to an implementing application), advertising content presented in an advertising provided in a broadcast or televised game's ad pods, advertising content physically provided within a sponsoring entity's retail location, or in online, print, email, social media post, or other mass media advertising, a product packaging, or advertising or event content displayed on, for example, a Jumbotron® (or similar display situated in public view at a live sporting event venue).

To accommodate limitations common to live venues, such as, for example, crowd noise that may diminish an ability of participating individual users to hear sounds emanating from mobile devices and limitations to cellular carrier or wireless signal service in a venue, varying combinations of content display via mobile devices to supplement public displays within the venue, and game element deployment via a combination of reliable SMS text messaging for query response selections, and bandwidth-dependent mobile applications for registration and leaderboard presentation may be deployed. This example encompasses but one combination of such connected and complementary services that may make the Squares Game or Super Squares Game information available to each participating individual user in a manner that is intended to make the gaming experience enjoyable and comparatively “hassle-free.”

Points may be awarded for correctly predicting or prognosticating an occurrence of a particular event in the game. Consider, for example, that points may be awarded for correctly predicting a pass or run, a score at a particular interval (or at a particular time in the event), attendance, or the like, or correctly recalling a portion of the preceding televised (or live, attended) game broadcast content. In embodiments, points may be awarded for a participating individual user opting into, or accepting, an offer presented by one or more of a game sponsor, a game host, a broadcasting network (including a TV network) or other involved entity.

In embodiments, such “opting in” may require participating individual users to join in a poll or survey presented by one or more of the above-listed entities. In such circumstances, point values may be assigned that are on par with point values that are associated with other activities of the participating individual user, or different from the point values associated with the other activities. The assignment of such point values may be at the discretion of one or more of the above-listed entities. In embodiments, the manner in which a particular participating individual user replies to any of the above stimuli may be afforded a “weighting” factor. Such an option may be considered once an entity determines that a benefit of weighting a particular manner of response does not serve, for example, as a disincentive to other participating individual users.

Automation of a Squares game according to the disclosed schemes allows for points, in an example leaderboard format, to be awarded for securing a game square that is partially correct at the end of a prescribed game, or game interval. An example would be where a participating individual user's game square matches only the home or away team score, but not both. As an example, a participating individual user secures, or is assigned, the game square 7/7. At the end of the game or game interval, the scores for the respective teams and in 7 and 3. The participating individual user may be awarded, for example, 3 points for the partial match, whereas a “perfect” match of both home and away team scores might offer the same or more points (such as, for example, 7 points). Such a hierarchy of awards is analogous to betting on horse races in which, for example, a quinella pays less than an exacta.

In embodiments, participating individual users may earn applied score multipliers. One manner by which such an applied score multiplier may be earned may be based on a participating individual user accomplishing one or more of (1) matching all (or the most) score combinations (potentially across a variety or plurality of individually-secured squares) for each specified round, (2) answering all (or the most) presented reactive questions correctly, (3) predicting all (or the most) specified outcomes, and (4) achieving a certain level of performance with respect to the user's fantasy player, squad or team selections. In embodiments, score multipliers may be awarded for each sequentially-consecutive pair or set of successful actions. In embodiments, an aggregation of score multiplier points may be accumulated according to match scores and/or matched answers, and a score multiplier may be applied for each of the participating individual users whose accumulated score multiplier points exceeds a pre-determined threshold.

Score multipliers may be applied at the end of a game or game interval, multiplying the aggregate of all points accrued by a multiplier to determine a participating individual user's ultimate final score for the game or the game interval. In a league-type or season-long scheme, score multipliers, or “league” (or “seasonal”) score multipliers, may be accumulated and applied for score matches, matching squares, consecutive round matches and the like over multiple live participation events. A notification scheme may be provided to advise participating individual users, at specified or random intervals, where a particular participating individual user's score stands among other participating individual users in the league-type or season long play. It should be noted that, in differing embodiments, score multipliers may be applied inclusive or exclusive of any tiebreakers.

In embodiments, a particular participating individual user's “score,” score multiplier or any tiebreaker may include, or otherwise take a form of, achieving a series of individual characters, which may be in the form of alphanumerics, emoticons, QR codes, images or the like, including combinations. Consider an example that, in a first round of a particular Squares Game, a participating individual user activity is presented indicating that a correct response (or even any response) will result in the award of an “S.” In a next round, a different participating individual user activity may be presented, the completion, or successful completion, of which may result in the award of a “C.” Over the course of the several rounds of the event, the participating individual user may have to collect the letters S-C-O-R-E, a team logo and a “smiley face” (see :-)) emoticon. Only upon achieving this combination of in-event awards may the player participant then receive an overall event award, receive an event score multiplier, or receive another reward. In embodiments, it is easy to see how the words “SCORE” may be replaced with an identifier associated with the sponsor, the team logo may be replaced with a sponsoring entity's logo and the like. The ability to use these Super Square in-event award identifiers to spell words and brands may provide opportunities to overlay sponsorship promotions. It may even be possible to allow participating individual users to select ALL Super Squares in advance of the start of an event, provided the selections spell a word, or brand, or some other restriction that provides participating individual users with the ability to pre-select their entries, but restricts the ability to necessarily select the Super Squares with the most favorable score combinations.

Using technology to access participating individual user registration demography, simulated virtual “tournaments” could be created, pitting subsets of participating individual users (e.g., according to any selectable demographic identifier, any region, a particular team affiliation, a particular brand association and the like). In this manner, a host of a tournament may be provided additional granularity as to the overall, or regional, demographics of participating individual users in order to, for example, determine insights into how subsets of a population of participating individual users may be faring with relation to one another. Such insight may be assist in providing appropriate feedback to, for example, sponsoring entities and better targeting their individually-sponsored activities for the participating individual users to a particular subset of the population of participating individual users.

Virtual league or tournament leaderboards may be system created according to a particular user entity's requirements. In this manner, information about the population of participating individual users, and identifiable sub-groups within that population of participating individual users may be tracked according to known BI and/or CI methods. Information reportable from the system according to demographics of the population of participating individual users may be sanitized of all particular participating individual user identifying information including, but not limited to, user names and other player-participant provided identification information that should be kept confidential and/or limited according to any participation agreement between, for example, a game host and individual participating individual users taking part in a particular game.

In embodiments, any display of scores of participating individual users, and the like, for comparison, would not need to provide actual identities of participating individual users. Multiple group subsets, or sub-groups, could be displayed (e.g., results of fans of all teams in a sports league, cities of a state, largest cities in the country, or according to other like indicators). The use of virtual tournaments (and reporting on same to the mass audience, via app, broadcast, other media, or some combination of some or all) may be considered to provide interesting content to some or all of the audiences. In embodiments, such virtual demographic leaderboards may provide the Super Square event host with an ability to target sponsoring entities in order to sell sponsorship rights in events associated with these virtual tournaments and leaderboards.

The disclosed embodiments use technology to provide a practical manner by which to handle the distribution of Squares Game matrices, as well as providing a flexible process of selecting and assigning the squares, along with aiding to collect any entry fees, tracking results, and distributing prizes, awards or rewards. In embodiments, a robust computing backend is provided to track populations of participating individual users and to provide the computing overhead to manage differing game constructs, subsets of populations of participating individual users, and awards/rewards schemes as are discussed in varying detail throughout this disclosure.

While the disclosed embodiments may incorporate components that apply to (and be substantially based on) conventional paper ten by ten matrix games, it is anticipated that popular implementations may be found in five by five matrix versions denoted by the darker lines and the letter annotations shown in the matrix participation area 110 in FIG. 1. Such an implementation may be discussed below as a “Super Squares” variant of the game.

“Pre-game” or pre-event Squares Game matrices may be provided in centralized locations, perhaps with URL's, unique letter designations, or QR codes inside of each square, which could allow participating individual users to enter a particular square's URL or unique letter into a web address or window of a supporting website or mobile application, or, for a participating individual user to select a square by scanning a square-unique QR code (on a displayed Squares Game matrix), or click the square on a display, which would allow the participating individual user to select that particular square.

In embodiments, the disclosed process, as enabled by hosting Squares Games electronic devices, may provide formatting options. For example, instead of randomly filling out the ten column and row “score heading” sections with random number from 0-9, the score heading(s) could be fixed, and the internal 25 or 100 matrix squares could instead be “jumbled” once the entry phase of the game is closed, and selections of squares are finalized. Such an electronic manifestation of the conventional paper Squares Game would be virtually impossible to accomplish in a conventional paper environment, but is relatively easily implementable in a virtual electronic-display environment.

According to the disclosed schemes, Super Squares games may provide a method by which a mass audience can participate in Squares Games, hosted by a network, sports venue, team, advertiser, individual, ad sponsor, event sponsor or the like. While traditional “sales” of squares may be enabled, it should be recognized that other “prize” schemes are implementable and may be preferable in certain jurisdictions and/or scenarios, particularly where it may be appropriate to avoid local ordinances controlling gambling. Due, for example, to the perceived degree or impact of luck (chance) required to win a game, and what is anticipated to become a very public nature of the game, it may be advisable, or even required in certain jurisdictions, to avoid charging for participating, and instead to offer other incentives (rewards) for participation, including entries (squares) in exchange for participating in reactive or ad-supported versions of the game, or by including “required” fantasy sports elements. In embodiments, the addition of skillful queries and questions, or fantasy sports elements, particularly as a final determining step of qualifying winners, may also be required in certain jurisdictions and according to applicable laws. Entry into the game itself may be controllable via a particular set of conditions established by any of the above-listed hosting-type entities.

When participating in Super Squares Games, jumbo 4×4 squares may be selectable by, or otherwise assigned to, participating individual users, five across horizontally and five up and down vertically (5×5), meaning that there are only 25 “Super Squares” to choose from, each representing 4 number combinations (4 times the number of score combos for participating individual users, and 4 times fewer squares to sell/fill). Each Super Square can be lettered A-Y (25 letters) or A-Z (with one letter of 26, from A to Z, being deleted), or representing a choice that essentially asks that the matrix square be randomized. Using letters makes it simple to select and record a square choice, and to share a particular participating individual user's square(s) choice with others as opposed to using the X and Y axis matrix address.

In embodiments, a game or event host could remove the “I” or “Q,” (the latter being shown in FIG. 1), to avoid the visual confusion of I with a number, or the letter “Q” with “O.” Separately, the letter Q may be used to select a randomized “Quick Pick” of a Super Square. In such a Quick Pick digital environment, those that select “Q” could be randomly assigned a letter, either before or after other interior squares are completed and/or jumbled.

A limitation of mobile phones is that a ten by ten annotated grid may be difficult to display and even more difficult to manipulate correctly on a small screen. If presented on a television or other larger format display screen, QR codes may be placed in each square. It is recognized that, in mobile screen applications, the QR codes could be too small or close together to be easily scanned without inadvertently scanning the wrong QR code. Therefore, a less error-prone method of allowing a participating individual user to select an interior square may be to refer to each square by a letter. A host could, therefore, use a lettering system to select a square (“Enter your letter HERE: ______”) as opposed to requiring the participating individual user to press a section of a screen, or scan a too-small QR code, thus providing an entry option that may eliminate some complexity and errors that are more likely in such examples.

In embodiments, another method of visual presentation may include skewing the squares matrix at a 45 degree angle, such that the score identifiers appear along any adjoining edges of a now diamond-shaped matrix. This visual presentation may be more ideally suited for displays that are viewed in a typical “landscape” mode, such as television displays, where the viewing area ratio is 4:3, or more commonly, 16:9 (width to height).

Because the digital display of the game's squares matrix is easily changeable, and as mentioned, a particular scheme can be implemented randomly to attempt to avoid a frequency with which participating individual users obtain unfavorable scores, or, for example, scores may be re-assigned t intervals, or between rounds. Separately, Super Squares Games can offer clusters of score headings that can implement the scheme by which to attempt to ensure that at least 1 of the 4 Super Squares score combinations is statistically more favorable than average. Also, rules of a Super Squares Game may require that either the interior Super Squares are jumbled once more each round, or, to ensure more variations, the audience of participating individual users can select a new letter each round, which is then jumbled.

Each sport can manipulate score headings that statistically increase a likelihood of securing at least one favorable combination of the four encapsulated by a participating individual user's Super Square. By way of example, each cluster of score headings could include a high frequency occurring score, and a lower frequency occurring score, based on statistical averages. In embodiments, Super Squares games associated contests may be broken into discernible rounds with three to six rounds potentially being specified per contest or live event. As scores tend to increase with time, in early rounds, having lower digit score combinations may be considered to be more favorable, where higher digit score combinations may be more valuable in later rounds (near the end of the underlying contest or sporting event, for example). This is particularly true of comparatively low scoring games like soccer, hockey and baseball (to an extent).

In embodiments, the score headings and assigned letter allocations may be permanently displayed, and a participating individual user may be assigned a random letter (and therefore, the Super Square and its related score combinations). This provides a consistent and simple means by which to quickly illustrate and distribute Super Squares, without a need to illustrate the “jumbling” process of score headings or squares. This may distribute at least one favorable interior square per Super Square.

As an example, a participating individual user with a Hockey Super Square “M,” may always receive the Home/Away score combinations of 2/2, 2/7, 7/2, and 7/7. Participating individual users could eventually become familiar with score combinations linked to each letter.

The disclosed Squares Games and Super Squares games may be sponsored by advertisers to (1) defray, avoid or supplement the cost of entry distribution, (2) advance logistics of hosting, registration, winner selection and participant notification, and/or (3) provide alternatives to monetary prize pools for non-monetary Squares Game and Super Squares game implementations. Squares Game and Super Squares game sponsors may provide advertising content that may be randomly interspersed with displays of the prize matrix at the sporting venue, or within a broadcast of the game. The advertisements may be made reactive in a manner that may be described for example in U.S. Pat. No. 6,606,745, the disclosure of which is hereby incorporated by reference herein in its entirety. Correctly responding to reactive queries may result in heightened attention (something every advertiser desires) and additional rewards to Squares Game and Super Squares game participating individual users.

Particularly for large mass audiences (such as televised programs) where millions of viewers (and potential participating individual users) may be viewing, the heightened attention paid to ads that are associated with the Squares Game and/or Super Squares game (denoted with an alert, mark or tone) may increase the value of an sponsor's advertisements, meriting a premium being paid by the sponsor. This premium may not only generate additional profits to the sponsor, sports league, broadcaster, post, or other participating entity but a portion of this premium might offset cost of conducting a mass Squares Game or Super Squares game event. This cost might also include the cost of a significant prize (such as a car), or an insurance premium to cover the cost of offering massive grand prizes.

Public offers and promotion of the Squares Game or Super Squares game, and an offer of more valuable prizes (new cars, or significant prize pools) may increase live venue attendance, and increase broadcast viewership of the sporting event (and therefore the audience for the advertisements). An increase in viewership, and attention paid to the advertisements (measurable through a reactive advertising concept) may increase a value of an advertisement inventory, further increasing the revenues generated from the advertisements. An increased revenue stream may be partially diverted back into prizing, creating a positive feedback “loop” that may increase prizes, then audience size, then prizes, and so on.

In embodiments, attendees at live venues (or watching TV broadcasts) may become participating individual users by selecting a Super Square letter 4-6 times or more over the course of the event, and watch a reactive ad before each “shuffle,” to qualify for a Super Square, if answered correctly. Failing to get the reactive question correct may result in the attendee not being registered as a participating individual user for that round, or as a participating individual user only retaining one of the four squares (perhaps the least desirable one in the combo).

The disclosed schemes for implementing a Squares Game or a Super Squares Game may be paired with fantasy sports, skill-based and prognostication elements to allow for sponsors to charge to play the games, or, they can be entirely subsidized by the venue, sponsors, or broadcast networks. The fantasy sports elements, or skill-based or prognostication questions may add tiebreaker elements. In embodiments, a “Jackpot Shot” question could ask, “What will be tonight's attendance,” or “How many shots on goal will be made this evening?” This differentiator could be used as a “closest to the correct answer” tiebreaker, or may be required to match the result precisely to win a particularly valuable (“super”) prize. Smaller prizes can be awarded each period/quarter/interval/increment, with a super prize being awarded to a participating individual user who wins all 4-6 periods, and, optionally, the Jackpot Shot.

As part of participating digitally, a participating individual user at a venue may be required to provide a seat number and name when registering, allowing the host to contact participating individual users (and to potentially film them using onsite cameras), integrating the Squares Game or Super Squares game into the live event as an added attraction. Similarly, on-air hosts might have real time access to the participating individual users and their ongoing entries, allowing them to be called or contacted on-air during the corresponding sporting event. This variation provides a real time, social media-friendly component to sporting events, which may be attractive to certain demographics.

In embodiments, a particular means of increasing interest and participation from all involved may be generally provided in increasing prize levels, which may be funded by sponsors particularly desiring greater direct connectivity with an entire population of participating individual users, or a particular sub-group of the overall population of participating individual users. According to the disclosed methods, all manner of deployment of additional branded integration may be provided from branded backgrounds for participation screens, to particular logos randomly placed or “popping up” on participation screens, to identification of prizes being provided according to particular branding.

In embodiments, participating individual users may need to visit a particular sponsors website and/or physical location in order to redeem or collect particular awards/rewards. According to these schemes, participating individual users may have access to larger Squares Game or Super Squares Game prizes pools, for themselves and also potentially for invited friends, by engaging in a particular activity by which a participating individual user may verify a visit to a sponsoring entity's physical and/or online properties. In a manner similar to the schemes described above for achieving an applying a scores multiplier, specific actions of individual participating individual users may elevate the standing of the individual participating individual users to, for example, a “Super Player,” permanently, or for the duration of a tournament, season, day, league, or individual event. Again here, participating individual user actions may include the scanning of a QR code, or the physical entry of a code into the Squares Game-or Super Squares game-enabled application, or the downloading of the sponsor's gaming or other application at a retail location, from an app store, or via a code or instructions provided on sponsor's website. This Super Player status may drive site and foot traffic in advance of the game, and also turn the physical or online properties into promotional locations. Sponsors'physical or online properties may even offer ability to upload the Squares Game-or Super Squares game application in a native version, or in a special version of the sponsor's own application Squares Game gamification capability enabled.

In embodiments, other related schemes may allow users to obtain a game clue, game points, or other items that would provide an advantage in a coming Squares Game or Super Squares game event, tournament, league, or season.

Embodiments may implement variations on this theme. For example, a Super Player who wins a Local Group or National Group event may lead to others in the Group (Super Players or standard player participants) to receive prizes that would not have otherwise been won by the standard participating individual users, but which are unlocked based on their relationship to the designated Super Player, and benefits bestowed through the sponsor, to “friends,” leaderboard group members, or others somehow “linked” to the winning or designated Super Player.

In embodiments, Super Players may receive special icon designations, which may appear next to their identifiers displayed leaderboards, and other displays. This may adds to interest in the sponsor, and due to the increased benefits (including an increased or separate prize pool or rewards available to Super Players), individual participating individual users without the Super Player icon may be invited, urged, or otherwise asked to visit the sponsor to become, or as a step in becoming Super Players themselves. In this manner, it is envisioned that peers will influence friends to take the necessary steps to “upgrade” to Super Player status as additional individual participating individual users acquiring such status may inure to the benefit everyone in a particular group of participating individual users, or overall.

In embodiments, information regarding Super Players, and the sponsor, may be provided to event hosts, sporting event venues, broadcast networks and the like in order that some recognition may be further provided to the status of participating individuals as Super Players in the Squares Games or Super Squares games associated with the event or broadcast. Such positive references may provide tangible promotional value to the sponsor, as well as elevating the positive commercial bond between event attendees, individual participating individual users, Super Players and the brand and brand sponsor.

In embodiments, once a leaderboard model according to the above-described scheme is adopted by a Squares Game or Super Squares Game host, and a number of player participants, has been established, the disclosed schemes may provide a capacity for ongoing daily, weekly or season-long leagues and tournaments to be supported.

In embodiments, a reviewable “standings” board may be displayable on individual mobile device screens or on display screens in public venues for displaying current standings in the league, season or tournament play of the public or private leagues. Groups of participating individual users may create their own Squares Game for Super Squares game leagues, or have periodic games (daily or weekly), which may even be defined across multiple sports disciplines.

In embodiments, certain head-to-head competitions may be specified so as to generate “wins” and “losses” for each team, group of individual participating individual users, or individuals themselves during the season. Where permitted, a capacity to implement a scheme for wagering may be provided. The disclosed schemes may enable a host to charge potential participating individual users for the opportunity to play, and, in addition to advertising revenues, can supplement the prize pool(s) with such entry fees.

In embodiments, prizes may incorporate Super Player prize premiums substantially in the manner described above, with paying and non-paying participating individual users, to provide different leagues based on prizing, or different prizes within one league based on the status of the designated winners.

In some professional and college sports leagues in the United States, multiple broadcast network feeds of games are provided to a national audience. Often, these broadcasts are ranked-ordered such that games most relevant to local markets are prioritized. For example, many professional football games commence at 1:00 PM EST Sunday, and again at 4:15 PM EST. Each feed may serve only one or several football games to any distinct market, so that the aggregated number of viewers, nationally, is significantly greater than in any one market. For example, a small market game between less followed teams may only draw 1.2 million viewers in two markets during the 1:00 PM EST slot, but the other 8 feeds, in aggregate, may attract 16 million viewers. Under this scenario, local groups may allow participating individual users to compete in real time within a local league, with the requirement that each participating individual user selects a local feed broadcast. Still, however, the participating individual user may be able to separately participate in a Squares Game teams in a non-local market, with both games being broadcast at approximately the same time. As it is unlikely that round terminations will be synchronized, the disclosed system and methods may provide timing synchronization across the games in order to allow each participating individual user to respond during their own defined “Check-In” period, with a group or national leaderboard not being shared with all users until after the last game and feed has reached a round completion or termination point.

In embodiments, a Squares Game or Super Squares game host may automatically aggregate all games into one or more national, regional or sectional leaderboards, increasing the available prize pool by aggregating varied revenue streams from multiple markets with regard to sponsorship (on a local, regional or national scale), ad and product placement (on a similar local, regional or national scale, Super Player premiums, and individual participating individual user entry fees, where unauthorized and appropriate).

In embodiments, aggregated prize pools may attract more viewers, who in turn may become additional Squares Game or Super Squares game participating individual users across multiple markets, cumulatively, having an effect of increasing advertising revenues and concomitant prize levels.

In embodiments, similar aggregation may occur across multiple live events in a same day. For example, participating individual users may participate in the 1:00 game and the 4:15 game, and may be provided an option to “cash out” after the 1:00 game, or to “let it ride” through the 4:15 game, and even through the 8:00 game, or even through a Monday game as a tiebreaker. Daily grand prizes and jackpots might be awarded to participating individual users with the highest score when aggregated across all games in a defined period.

In embodiments, aggregated “scores” may even be collected from multiple sports over a defined period, particularly when sport seasons overlap (such as when there are hockey, football, and basketball games occurring within the same day or week).

In embodiments, for example, each March, potential participating individual users gather to individually complete their brackets prior to the commencement of a 64+ team tournament. Potential participating individual users may predict the outcome of each matchup, over a series of predicted pairings reducing the field progressively to 32, 16, 8, 4, 2 and 1 overall winner, over 6 rounds and several weeks. A reasonable estimate for a single participating individual user to predict correctly all game outcomes in advance of the tournament, a feat which has never been reported to date is 1 in more than 100 billion.

Embodiments according to the disclosed schemes may adapt a Squares Game to the prognostication thrill of bracket selections in advance. The fantasy sports selections, quizzing, prognostication, and rewarding matching score elements of the Squares Game or Super Squares game may be adapted to allow individual participating individual users for each bracket round (e.g., the opening round of 32 games) an opportunity to select one game and predict the outcome of the game (winner, and optionally, tiebreakers, as discussed below).

In embodiments, the disclosed schemes may afford a participating individual user an opportunity to select multiple games over certain periods such as, for example, an early Saturday game and a late Saturday game. The Squares Game host may enable a Squares Game in which a new square is allocated each round in a particular game, or to remain constant for the duration of the game. Participating individual users may be afforded an opportunity to select the squares.

In embodiments, participating individual users may be required to select a single game and winner for each round in advance of the commencement of the tournament, as opposed to selecting a winner of every game and subsequent round.

In embodiments, participating individual users may be afforded an opportunity to select a new winner for each round, once prior round results are concluded and a list of the remaining teams from which the player participant may select is established. This variation may mediate the risk to a participating individual user in attempting to select winners in later rounds for which the participating individual user may have already been eliminated. Given the capacity and adaptability of the Squares Game concept to variations in selection, scoring, cumulative scoring and prize awarding, the “bracket” version may include an automated implementation that permits participating individual users to select only among games of their choosing, selecting a winner, and also permitting participating individual users to select squares to coincide with a particular round, in a manner that may be pre-determined by the host, or by the participating individual user. By way of example, the automated scheme may provide a capacity to collect information from participating individual users regarding a selection of a game of their choice, a prediction of the winner, and a selection of any square of their choosing for any round of their choosing.

In embodiments, the disclosed automated schemes may otherwise require that a square be selected for a final round in a particular game. This variation may permit a participating individual user to select a single square for each round, in advance.

Each of the variations of the disclosed automated Squares Game applied in a bracket context may afford participating individual users a considerable amount of control over their entries even as the tournament progresses. Participating individual users, for example, may associate squares with their individual selections and prognostications for specific games, winning teams, and squares relative to the final score, or for any round or rounds.

In embodiments, this level of interaction and control by participating individual users may provide a favorably perceived condition relative to the involvement of large groups of participating individual users, and may provide an advantage to participating individual users who pay closer attention to the actual progress of the games, teams, and players (including those participating individual users'fantasy player/squad/team selections) involved in the turn of allowing the participating individual users to exercise their knowledge when selecting players, games, outcomes, and squares and related score combinations, even as unexpected occurrences arise in the progress of the tournament.

An advantage of the “bracketology” Squares Games or Super Squares games is that these schemes combine interplay with audiences attracted to both games while simplifying the qualification process, thereby providing ability to attract a wider audience.

The automation of the Squares Game in the disclosed adaptable format may also provide flexibility for a host to create and implement a rules structure prior to commencement of the tournament that effectively employs known or calculable statistical measurements of certain events occurring, in order that the likelihood of winning according to this scheme is significantly easier than selecting a perfect bracket according to the traditional method. Also, the use of aggregated Squares Game points and leaderboards provides all participating individual users, over the course of the tournament, with the ability to remain “in the game,” even in the event that a particular participating individual user may have, at some point over the course of the tournament, fallen out of contention for the jackpot or mega jackpot prize pool.

It should be recognized that games according to the disclosed Squares Games or Super Squares gamification schemes may provide an automated platform for participating individual user interaction that may attract millions of participating individual users over a widely viewed sporting event or tournament of games, it is possible that there may be multiple participating individual users with identical point scores, and even with “Perfect Super Square Matches” over a period of time (such as after four or six rounds of Super Squares). To avoid use of a random drawing to break ties, pre-determined and/or pre-planned participating individual user selectable tiebreaker tools may be automated. In embodiments, one category of such tiebreaker tools may incorporate a particular player participant skill set exemplified in the Squares Game or Super Squares Game. These may include prognostication, ad attentioning, and/or content attentioning.

In embodiments, one or more prognostication questions may be derived and presented to participating individual users at some specified, or random, time prior to the event. Tiebreaker points may be awarded during the event then based on how close a participating individual user's responsive estimate comes to the actual correct answer. The number of maximum points (say, 3 points) would be awarded based on either a proximity or accuracy. By way of example, prior to a football game, each participating individual user may be asked to estimate a total number of yards to be gained by a particular running back. A maximum allowed variance may be set at, for example, 40 yards, plus or minus. If a particular participating individual user's prognostication misses the actual number of yards gained by a particular running back by 4 yards, the particular participating individual user may be deemed to have gotten the answer 90% correct. In such an instance, the particular participating individual user may be awarded 9 of 10 available tiebreaker points. The prognostication events, variations, and available points may be set by the host prior to the game via an administrative portal accessible by the host to modify the point schemes for the Squares Game or Super Squares game to which the football game is linked. The tiebreaker scheme may be applied over any number of other tiebreakers, and result in an aggregated resulting in a total tiebreaker score, which can be added to a total of a player participants accrued points, modified according to any multipliers, when appropriate and only to break ties in 2 or more participating individual users'aggregate scores to determine a single winner.

In embodiments, employing combinations of predictions, reactive quizzes, or square matching elements could provide a robust description of a game of skill that would qualify an automated Squares Game for wagering deployment. It is recognized, however, that broad deployment of such an automated Squares Game may be hampered in that some jurisdictions may require gaming licensure consistent with prop bets or parlays if only these elements are employed. To avoid these impediments to entry in certain jurisdictions, exemplary embodiments of an automated Squares Game may include elements of fantasy sports including selection or drafting of players for a mock or fantasy team or squad. By supplementing predictive and quiz elements in this manner, such exemplary embodiments with fantasy sports elements including selection of players or team units are intended to clearly avoid jurisdictional restrictions.

In embodiments, a participating individual user may be allocated a salary cap of a number of salary units to be used to select a number of players, which may be a combination of particular position players, for example: a quarterback, a running back, a wide receiver/tight end, a kicker, and a number of players in a defensive unit. The participating individual user may select their roster as a prerequisite to being assigned a first random single-combination square or a four-combination Super Square or to selecting/purchasing the first square or Super Square. In embodiments then, once “enrolled” through the fantasy sports process, subsequent squares or Super Squares may be assigned to, or selected/purchased by, participating individual users pursuant to the rules of the game being played.

In embodiments, participating individual users may be enabled to slide, jump, or otherwise alter the square or Super Square combinations assigned or selected/purchased at the commencement of each event or event interval. In embodiments, participating individual users may use saved salary cap units (instead of, or in addition to making a wager or cash payment) in order to be eligible to alter the square locations. In embodiments, ultimately unspent salary units may form a multiplier or bonus for the total score achieved, or may simply be added to the final overall score, or may be used even as a tie-breaker in the event one or more participating individual users are tied.

In embodiments in which an objective is to award only one grand prize to only one participating individual user, the tiebreaker scheme may be particularly rigorous according to combinations of the above elements. Variations and prognostications may be weighted, multiple part responses may be used (including answering one or more characteristic questions regarding the event, the broadcast, the event or the like) and/or a hierarchy of correct responses and/or prognostications may be established. The disclosed schemes may provide virtually unlimited opportunity for a host to locally or regionally modify the rules in any manner that affects awards/rewards. Important to the fidelity of the implementation of any Squares Game or Super Squares game according to this disclosure will be in ensuring that participating individual users are advised and/or informed of the rules prior to the commencement of play. In this regard, the disclosed systems methods may provide standard sets of instructions and standard deviations from those instructions that participating individual users will be required to acknowledge prior to commencing a particular game, group of games, league and/or tournament.

In embodiments, a score based on predictions, for example, may only be utilized in the event a tie between participating individual users has occurred, or the one or more predictions may always be included in the final score calculation for all participating individual users. In the event the prediction element is always included to arrive at a final score, the maximum score value for perfect predictions may be nominal when compared to the points associated with fantasy draft points, and square matching points.

The disclosed schemes may also provide matrix-based selections that are not “scores” based at all, or that do not require the provision of “scores-based” matrices, in multiple variations and schemes that may be optimized to particular sporting events.

In embodiments, a host may allow or suppress the consecutive requirement. In such an example, a requirement may simply be, “Collect 4 or 5 winning Super Squares over the course of this event.” Alternatively, the host may provide one or more “mulligans,” or “do-overs” per event, such that, while the consecutive winning Super Square requirement may remain intact, a participating individual user may receive, earn, or purchase a “mulligan” that may be electively used to convert a losing round into a winning round.

In embodiments, other means of increasing a likelihood of winning would be to allow some or all participating individual users to select their own Super Square combination in advance of a round, such that participating individual users with a thorough knowledge of the game might have an increased likelihood of selecting a winning combination. A “Pick Your Square” bonus may be awarded randomly (such as to those who are awarded the letter “Q”), or may be otherwise received, earned or purchased. For example, in a low scoring game like hockey, where scores are often 0-0 after the first period, selecting the Super Square containing a “0-0” combination would increase a number of winning Super Squares in that round.

In embodiments, a host may increase a likelihood of winning by further reducing the number of Super Squares. The host may elect, for example, to create only 5 Super Squares, in which a shape of each cluster of twenty combinations in a ten by ten matrix may deviate from a true “square” shape. In embodiments, a host may also add a “common zone” within the matrix that may be similar to a center square of a Bingo card, which is filled for all participating individual users. The host may, for example, purposefully allocate frequent or infrequent scores to the shared zone to further impact the frequency of winning Super Square combinations.

In embodiments, the disclosed schemes may be adaptable to sporting events where numbers are frequently used, but for other than traditional scores. For example, in many racing events, there are no “scores,” merely finishing outcomes/orders (first, second, or third.) Numbers, however, are worn by players or event participants (such as in professional motor sports, like auto racing, in which the numbers are displayed on the cars/trucks/motorcycles).

By way of comparison, “score headings” may be replaced with “driver/car numbers” and “Home” and “Away” might be replaced with “First Place” and “Second Place,” for the event where Super Squares may be awarded at lap intervals. In such an implementation, for a 300 mile race, Super Squares may be awarded every 50 laps, totaling 6 intervals. By way of example, at the 50 lap mark, the first place car might bear the number “17,” and the second place car might bear the number “3,” meaning that the holder of the Super Square “7,3” would have a winning super square.

A racing-inspired Super Squares game may have applications to other forms of racing, including motocross, and horse racing, and separately to non-racing events like jai alai and other sports where numbered event participants are ranked based upon the finish.

Finally, for events where finishes are ranked numerically, but where the event participants are not numbered visually (i.e. golf tournaments), a host could assign a number to each event participant in advance of the event (as in a golf tournament, where upwards of 100 golfers compete). So, in such an example, the top finishing golfer may assigned #42, and the second place finisher may be golfer #2, the participating individual user holding the “2,2” Super Square over a certain interval (i.e., after the first round, or first 9 holes) would be a winner.

The disclosed embodiments represent non-limiting examples of variations to the Squares Game and Super Squares games implementing many variations and encompassing “scores-based” games, and “non-scores-based” games. Further, the totality of the above discussion advises multiple variations on the traditional or conventional paper Squares Game in order to modify the game in a manner that is commensurate with a desired level of participation and/or modify the game in a manner that properly allocates awards/rewards, and a potential for multiple winners, according to an available prize pool in consideration of the actual level of participation. Finally, the disclosed schemes are intended to advantageously integrate, in an automated manner, attractive attributes of the basic conventional paper Squares Game, fantasy sports elements, prognostication, availability of wagering (where authorized), and attractiveness in the marketplace to participating individual users and also to potential sponsoring entities.

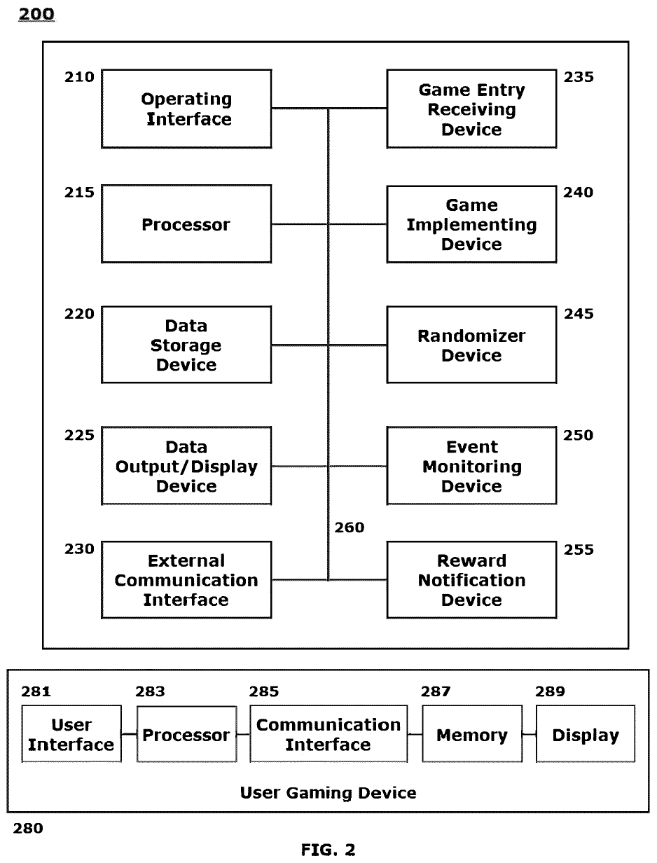

FIG. 2 illustrates a block diagram of an exemplary system 200 for implementing an automated Squares Games according to this disclosure.

The exemplary system 200 may include an operating interface 210 by which a user as a host may communicate with the exemplary system 200. The operating interface 210 may provide a host an opportunity to initiate the automated Squares Game and to input parameters appropriate to the conduct of the automated Squares Game in the manner outlined above, including parameters for entry criteria including fantasy sports player or team selections, aggregation of discrete Squares Games and establishment of tiebreakers, all of which may be user-selectable by the host. The operating interface 210 may be configured as one or more conventional mechanisms common to computing and/or communication devices that may permit the host to input information to the exemplary system 200. The operating interface 210 may include, for example, a conventional keyboard, a touchscreen with “soft” buttons or with various components for use with a compatible stylus, a microphone by which the host may provide oral commands to the exemplary system 200 to be “translated” by a voice recognition program, or other like device, including any wearable I/O device, by which a user may communicate specific operating instructions and pre-determined parameters setting to the exemplary system 200.

The exemplary system 200 may include one or more local processors 215 for individually operating the exemplary system 200, and for carrying into effect the disclosed schemes in the exemplary system 200. The processor 215 may carry out routines appropriate to operation of the exemplary system 200, and may undertake data manipulation and analysis functions appropriate to the implementation of the Squares Game in any and all of the above-noted variations including, but not limited to, aggregation and/or leaderboard tracking. Processor(s) 215 may include at least one conventional processor or microprocessor that interprets and executes instructions to direct specific functioning of the exemplary system 200, and control of the automated Squares Game implementation in any of the variations from a mirror of the conventional paper Squares Game, modified as a Super Squares Game, or varied to include aggregation, leaderboard tracking and/or tiebreakers according to this disclosure.

The exemplary system 200 may include one or more data storage devices 220. Such data storage device(s) 220 may be used to store data or operating programs to be used by the exemplary system 200, and specifically the processor(s) 215 in carrying into effect the various participating individual user interacting, game displaying, leaderboard tracking, aggregation, Super Player identification and all forms of rewards notification functions of the disclosed Squares Game and Supers Squares game schemes according to this disclosure. At least one of the data storage device(s) 220 may be used to store the gamification application and to temporarily store in-process Squares Game matrix display information. At least one of the data storage device(s) 220 may be used to store particular identification information that may be collected incumbent to participating individual users requesting to play the game, or to register for particular league, tournament, season or selection of events. Such data storage device(s) 220 may also include cumulative scoring related to participation of each participating individual user according to any one of the implemented gamified scenarios.

The data storage device(s) 220 may include a random access memory (RAM) or another type of dynamic storage device that is capable of storing updatable database information, and for separately storing instructions for execution of system operations by, for example, processor(s) 215. Data storage device(s) 220 may also include a read-only memory (ROM), which may include a conventional ROM device or another type of static storage device that stores static information and instructions for processor(s) 215. Further, the data storage device(s) 220 may be integral to the exemplary system 200, or may be provided external to, and in wired or wireless communication with, the exemplary system 200, including as cloud-based storage components.

The exemplary system 200 may include at least one data output/display device 225, which may be configured as one or more conventional mechanisms that output information to a user, in this case a host, on a progress of the Squares Game. The data output/display device 225 may be used to indicate to the host information regarding a compilation of the matrix for a particular game, as well as a progress of a live event with which the Squares Game is associated. It is not necessary that the host monitor the actual conduct of the automated Squares Game by the exemplary system 200, but the host is afforded that option. Further, the data output/display device 225 may be in the form of a printer or other data delivery means by which the “rules” of a particular game may be provided to each individual player participant, particularly as those “rules” may be modified by the host for a particular playing of the game.

The exemplary system 200 may include one or more separate external communication interfaces 230 by which the exemplary system 200 may communicate with one or more offboard Squares Game implementing components including, but not limited to, an external display, and any user gaming device 280 on which a participating individual user may choose to play the game, which may be in wired or wireless communication with the exemplary system 200. It is anticipated that components of the exemplary system 200 may communicate with a broad spectrum of displays and user gaming devices 280 that may generally be employed by each participating individual user in execution and monitoring of the each participating individual user's participation in the Squares Game, tournament, league, event or the like.

The exemplary system 200 may include a game entry receiving device 235 that may be used to receive and store individual player participant registration/identification information for participating individual users seeking to participate in a particular Squares Game. The prospective participating individual user may have to identify the live event to the exemplary system 200 with which the prospective participating individual user may choose the Squares Game to be associated.

In addition to receiving prospective player participant registration/identification information, the game entry receiving device 235 may be usable to interact with the participating individual user's gaming device 280 to receive a entries of fantasy sports elements and selections as entry conditions (where appropriate) and selection of one or more of the plurality of squares in the game matrix for the particular Squares Game in which the participating individual user intends to participate. Participating individual user selection of the one or more of the plurality of squares in the game matrix may be according to any of the above discussed methods, including all manner of automated means or participating individual user manipulation of a user interface associated with the participating individual user's gaming device 280.

The exemplary system 200 may include a game implementing device 240 that may execute functions for carrying into effect the Squares Game according to the disclosed schemes, and as modified by the host, in the exemplary system 200. The game implementing device 240 may itself be a function of the processor 215, or may exist in, or associated with, the exemplary system 200 as a stand-alone component.

The game implementing device 240 may accept input from the game entry receiving device 235 and a randomizer device 245, which may generate the random number schemes described above, to generate and monitor the status of the game matrix through the duration of the live event with which the Squares Game of the exemplary system 200 is associated.

The game implementing device 240 may also be usable to undertake the prescribed aggregation scheme across multiple games, or across multiple events, and otherwise to implement and track the disclosed leaderboard scheme.

The exemplary system 200 may include an event monitoring device 250 that may be usable to monitor the progress of the live event with which the in-process Squares Game is associated. At prescribed intervals (rounds), the game implementing device 240 may receive inputs from the event monitoring device 250 regarding, for example, a score, or other progress, of the live event and may begin a determination scheme by which to determine whether any participating individual user may be declared a winner for each specified round in the live event.

The exemplary system 200 may include a reward notification device 255 by which, when a participating individual user is determined to have won a prize, award, reward or the like, in the form of, for example, merchandise, discounts, coupons, cash and/or other incentives, the participating individual user may be immediately notified. Like the game implementing device 240 above, the reward notification device 255 may be a function of the processor 215, or a stand-alone device, either of which may present reward information user gaming device 280 to be displayed, for example, on a display 289 of the user gaming device 280. The reward notification device 255 may be usable as well to advise a participating individual user as to a status as a Super Player substantially in the manner outlined above.

The exemplary system 200 may communicate with one or more user gaming devices 280, each of which may include a user interface 281, a processor 283, a communication interface 285, a memory 287, and a display 289. A participating individual user in a Squares Game or Super Squares Game may employ a user gaming device 280 for interaction with the exemplary system 200 as it carries into effect the disclosed schemes for implementing the Squares Game or Super Squares game. It should be noted that one or more of the user gaming devices 280 by which a participating individual user may participate in the Squares Game or Super Squares game may be, in addition to the many devices catalogued above, user-wearable I/O and/or computing devices such as, for example, wearable computer/communicating display glasses and/or watches, biometric sensors, virtual reality (or immersion) devices including goggles, helmets, tactile gloves and the like, and other known or developed wearable components for carrying out one or more of computing and/or communicating functions allowing a participating individual user to communicate with the exemplary system 200.

All of the various components of the exemplary system 200, as depicted in FIG. 2, may be connected internally, and to one or more external components by one or more data/control busses 260. These data/control busses 260 may provide wired or wireless communication between the various components of the exemplary system 200, whether all of the components of the exemplary system 200 are housed integrally in, or are otherwise external and connected to the exemplary system 200.

It should be appreciated that, although depicted in FIG. 2 as an essentially integral unit, the various disclosed elements of the exemplary system 200 may be arranged in any combination of sub-systems as individual components or combinations of components, integral to a single unit, or external to, and in wired or wireless communication with the single unit of the exemplary system 200. Wireless communications may be by RF radio devices, optical interfaces, NFC devices and other wireless communicating devices according to RF, Wi-Fi, WiGig and other like communications protocols. In other words, no specific configuration as an integral unit, or as a support unit, is to be implied by the depiction in FIG. 2. Further, although depicted as individual units for ease of understanding of the details provided in this disclosure regarding the exemplary interface and control system 200, it should be understood that the described functions of any of the individually-depicted components may be undertaken by one or more processors 215 connected to, and in communication with, one or more data storage device(s) 220.

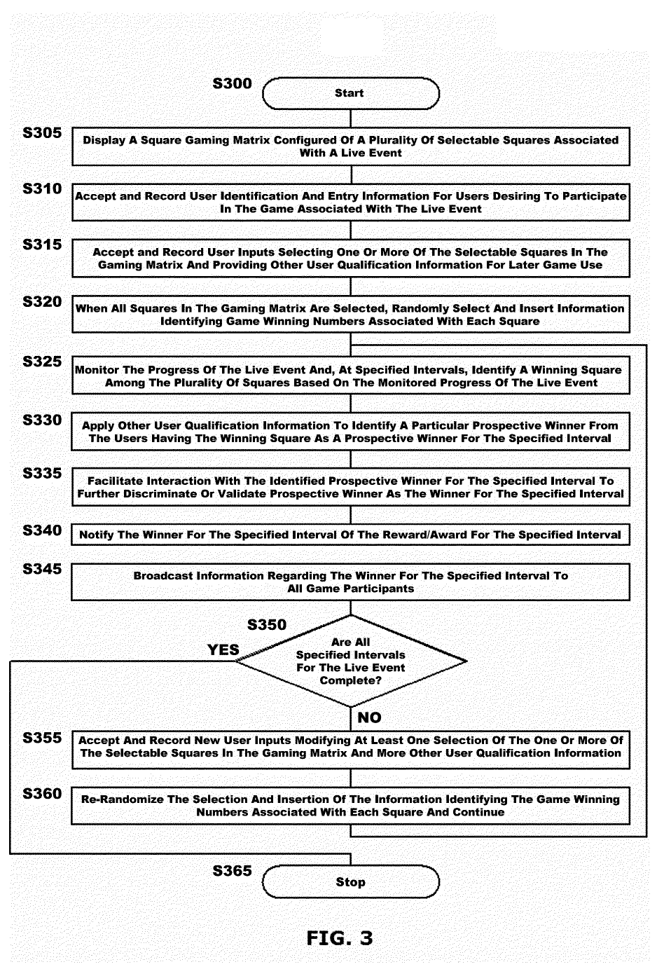

The disclosed embodiments may include exemplary methods for implementing automated Squares Games. FIG. 3 illustrates a flowchart of a first exemplary method. As shown in FIG. 3, operation of the method commences at Step S300 and proceeds to Step S305.

In Step S305, a gaming matrix configured of a plurality of selectable squares associated with a live event may be automatically caused to be displayed. Operation of the method proceeds to Step S310.

In Step S310, player participant identification information for the participating individual user desiring to participate in the game associated with the live event may be accepted and recorded. Operation of the method proceeds to Step S315.

In Step S315, player participant input selecting one or more of the selectable squares in the gaming matrix may be accepted and recorded. Operation of the method proceeds to Step S320.

In Step S320, when all of the squares in the gaming matrix are selected by, or otherwise identified with, a participating individual user, information identifying game-winning numbers associated with each square may be randomly inserted. The process may be automated to replicate the look and feel of a conventional matrix by randomly filling orders squares with selected numbers. Otherwise, the border numbers may be fixed and the participating individual user-selected squares maybe randomized. Operation of the method proceeds to Step S325.

In Step S325, the progress of the live event may be monitored. At specified intervals (rounds), numbers associated with the live event may be extracted to identify a winning square for the specified round among the plurality of squares. This may be the only, or otherwise simply a first, step in identifying a winner of the Squares Game for the specified round. Operation of the method proceeds to Step S330.

In Step S330, other qualification criteria may be applied to identify prospective winner for the specified round. This may include additional qualifying information, or additional random information, or tiebreakers, or identification as a Super Player, or an aggregation and/or leaderboard scheme, or any one or more of the indicated schemes described in detail above. Operation of the method proceeds to Step S335.

In Step S335, a prospective winner may be presented with a reactive advertising scheme in which the prospective winner is presented with advertising content and then asked a series of questions regarding that advertising content. A prospective winner's ability to correctly answer questions based on the advertising content may be the last step in qualifying the prospective winner as the winner for the specified interval. Operation of the method proceeds to Step S340.

In Step S340, the winner for the specified round may be notified of the reward or award for which the winner has qualified. Additional instructions may be provided, for example, to indicate to the winner how to redeem or otherwise collect the reward or award for which the winner has qualified, or in an accumulated gaming scheme, a leaderboard entry may be provided. Operation of the method proceeds to Step S345.

In Step S345, information regarding the winner for the specified round (or standings on a leaderboard) may be broadcast to all game player participants. Operation of the method proceeds to Step S350.

Step S350 is a determination step in which a determination is made as to whether all of the specified rounds, games, events in a tournament, league events or the like, associated with the Squares Game are complete.

If, in Step S350, a determination is made that all of the specified rounds, games, events in a tournament, league events or the like associated with the Squares Game are complete, operation of the method proceeds to Step S365, where operation of the method ceases.

If, in Step S350, a determination is made that all of the specified rounds, games, events in a tournament, league events or the like associated with the Squares Game are not complete, operation of the method proceeds to Step S355.

In Step S355, player participant inputs modifying at least one selection of the one or more of the selectable squares in the gaming matrix may be accepted and recorded, as allowed. Operation the method proceeds to Step S360.

In Step S360, the selection and insertion of information identifying game-winning numbers associated with each square may be re-randomized. Operation of the method reverts to Step S325.

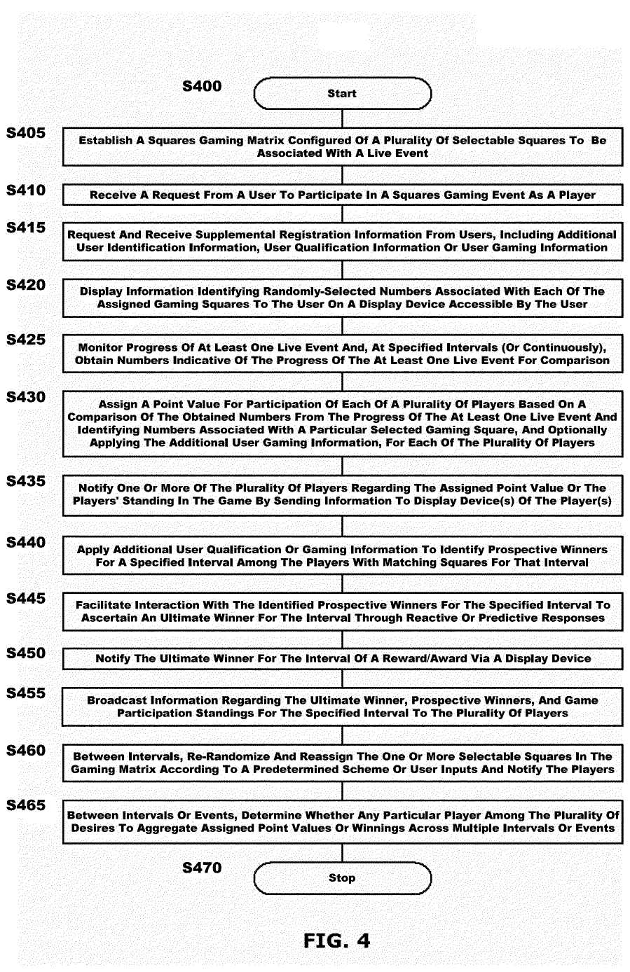

FIG. 4 illustrates a flowchart of a second exemplary method. As shown in FIG. 4, operation of the method commences at Step S400 and proceeds to Step S405.

In Step S405, a gaming matrix configured of a plurality of selectable squares associated with a live event may be established by a system. Operation of the method proceeds to Step S410.

In Step S410, a request from a potential participating individual user to participate in a gaming event as a player may be received. Operation of the method proceeds to Step S415.

In Step S415, supplemental registration information for the prospective participating individual user may be requested and received from the prospective participating individual user. Such supplemental registration information may include, for example, information further identifying the prospective participating individual user and/or providing some basis for qualification of the prospective participating individual user to participate in the gaming event. As an example, a prospective participating individual user may be required to make certain fantasy sports player(s), squad(s) or team(s) selections.

One example of such a fantasy sports selection scheme may proceed as follows:
a. The prospective participating individual user may be allocated a salary cap of, for example, sixteen salary units.
b. The prospective participating individual user may select five (5) players from all players participating in a pro football game that week: one (1) Quarterback, one (1) Running Back, one (1) Wide Receiver/Tight End, one (1) Kicker, and one (1) Defensive Unit.
c. Selections may be constrained by maximum salaries assigned to the best projected players in each position category, e.g., five (5) salary units, with the least being one (1) unit, and all eligible active players being priced between this maximum and minimum (including even to decimal places, e.g., 3.75 points for a particular quarterback).
d. The prospective participating individual user may select a roster, after which the prospective participating individual user may be assigned a first random single-combination square or a four-combination Super Square, or the prospective participating individual user may be permitted to select/purchase the first square or Super Square as a now-registered/cleared participating individual user for that game. In embodiments, then subsequent squares or Super Squares may be assigned, selected or purchased pursuant to the rules of the game being played.
e. To reduce or prevent the likelihood of a tie, the host may also require each participating individual user to answer one or more predictive questions, which would be scored based on a formula or algorithm that would provide a higher score based on proximity to the final outcome, when compared to the predictions. The score based on the prediction may later be used in the event of a tie between two or more participating individual users has occurred. The one or more predictions may be included in the final score calculation for the participating individual users. In embodiments in which a prediction element is included to arrive at a final score, the maximum score value for perfect predictions may be nominal when compared to the points associated with fantasy or draft points, and square matching points.
f. In embodiments, participating individual users may slide, jump, or otherwise alter a square or Super Square combination assigned or selected at the commencement of each event round or interval of the game. Saved salary cap units may be used (instead of, or in addition to, making a wager or cash payment) in order to be eligible to alter the square locations. By way of example, as an incentive to save salary cap units, for each salary cap unit not invested in a player salary, the participating individual user may be allowed one square “slide” per interval (or sporting event). Should the participating individual user be permitted a salary cap budget of 20 units, but require 1 salary cap point to slide a Super Square once each quarter, by spending 16 or fewer points on player salary, the saved 4+ units would permit the participating individual user to make 4 slides over the course of the event.
g. In embodiments, saved salary cap units may otherwise be applied as a discount to the cost of the square slide. For example, the cost to make a slide may be $5, but the participating individual user, having saved 3 salary units, may be permitted to exchange salary units for dollars, thereby being able to pay only $2 to move/slide a square.
h. In embodiments, unspent salary cap units may form a multiplier or bonus for the total score achieved for the game. For example, should the participating individual user save 2.3 units of the 20 units of salary available, the 2.3 units might be applied as a multiplier to one or more of the scores achieved by drafted players, or as a multiplier for one or more squares or Super Squares matched over the duration of the game. Otherwise, unspent salary cap units may be added to the final overall score for the game, or may be used even as a tie-breaker in the event two or more participating individual users are tied.

Collected entry information, including fantasy information, from prospective participating individual users may be used to register the prospective participating individual users, and may also be stored for later use, for example, as tiebreaker information. Such information may also include input from the user regarding a number of specific intervals in a particular live event that the user chooses to participate as a player, and/or a number of particular live event in which the user chooses to participate as a player. Operation of the method proceeds to Step S420.

In Step S420, information identifying numbers associated with each of the assigned gaming squares may be displayed to the participating individual user on a display device accessible by the participating individual user. The display device may be, for example, a common display device in a particular event venue, or it may be a participating individual user's own portable mobile computing and/or communicating device, or virtually any other like display device by which information may be presented to the participating individual user. Operation of the method proceeds to Step S425.

In Step S425, the progress of the live event may be monitored. Constantly, or at specified intervals (rounds), numbers indicative of the progress of the live event may be obtained. Operation of the method proceeds to Step S430.

In Step S430, a point value for participation of each of a plurality of participating individual users may be assigned based on a comparison of the obtained numbers from the progress of the at least one live event and identifying numbers associated with a particular selected gaming square for each of a plurality of participating individual users. An assigned or accumulated point value may represent an only, or otherwise simply a first, step in identifying a winner of the Squares Game during and/or for the specified round. Operation of the method proceeds to Step S435.

In Step S435, one or more participating individual users may be notified regarding the currently-assigned point values based on information being sent to a display device. Operation the method proceeds to Step S440.

In Step S440, additional qualification may be applied to identify prospective winner for the specified round. This may include additional qualifying information, such as predictions or fantasy selections of each of the participating individual users identified as prospective winners, or other additional random information, or other tiebreakers, or identification as a Super Player, as discussed above, or an aggregation and/or leaderboard scheme, or any one or more of the indicated schemes described in detail above. Operation of the method proceeds to Step S445.

In Step S445, a prospective winner may separately be presented with a reactive advertising scheme in which the prospective winner is presented with advertising content and then asked a series of questions regarding that advertising content. A prospective winner's ability to correctly answer questions based on the reviewed advertising content may be a step in qualifying the prospective winner as the winner for the specified interval. Operation of the method proceeds to Step S450.

In Step S450, the winner for the specified round may be notified of the reward or award for which the winner has qualified. Additional instructions may be provided, for example, to indicate to the winner how to redeem or otherwise collect the reward or award for which the winner has qualified, or in an accumulated gaming scheme, a leaderboard entry may be provided. Operation of the method proceeds to Step S455.

In Step S455, information regarding the winner for the specified round (or standings on a leaderboard) may be broadcast to all game player participants. Operation of the method proceeds to Step S460.

In Step S460, the one or more selectable squares in the gaming matrix may be reassigned between intervals, and the participating individual users notified. Operation of the method proceeds to Step S465.

In Step S465, a determination may be made between intervals and/or events as to whether a particular participating individual user desires to aggregate point values and/or winnings across multiple intervals and/or events. Operation of the method proceeds to Step S470, where operation of the method ceases.

The disclosed embodiments may include a non-transitory computer-readable medium storing instructions which, when executed by a processor may cause the processor to execute all, or at least some, of the steps of either of the methods outlined above.

The above-described exemplary systems and methods reference certain conventional components to provide a brief, general description of suitable operating and presentation scheme implementing environments in which the subject matter of this disclosure may be undertaken for familiarity and ease of understanding. Although not required, embodiments of the disclosed systems and methods may be provided, at least in part, in a form of hardware circuits, firmware, or software computer-executable instructions to carry out the specific functions described. These may include individual program modules executed by processors.

Those skilled in the art will appreciate that other embodiments of the disclosed subject matter may be practiced in myriad configurations for effecting the disclosed Squares Game schemes with applications hosted on a spectrum of computing and communicating devices.

As indicated above, embodiments within the scope of this disclosure may include computer-readable media storing computer-executable instructions or data structures that can be read and executed by one or more processors for controlling the presentation processes for gaming matrices according to the disclosed schemes, and for carrying into effect the overall gaming schemes. Such computer-readable media can comprise RAM, ROM, EEPROM, CD-ROM, flash drives, data memory cards or other analog or digital data storage devices that can be used to carry or store desired program elements or steps in the form of accessible computer-executable instructions or data structures.

Computer-executable instructions include, for example, non-transitory instructions and data that can be executed and accessed respectively to cause a processor, for example, in an automated Squares Game implementing device or system to perform certain of the above-specified data acquisition, game implementation, and display and reporting functions. Computer-executable instructions may also include program modules that are remotely stored for access and execution by a processor.

The exemplary depicted sequences of executable instructions or associated data structures represent examples of corresponding sequences of acts for implementing the functions described in the steps of the above-outlined exemplary methods. The exemplary depicted steps may be executed in any reasonable order to carry into effect the objectives of the disclosed embodiments. No particular order to the disclosed steps of the methods is necessarily implied by the depiction in FIG. 3 or 4, except where a particular method step is a necessary precondition to execution of any other method step. Separately, not all of the depicted steps of the methods shown in FIGS. 3 and 4 need to be implemented in any particular embodiment.

Although the above description may contain specific details, they should not be construed as limiting the claims in any way. Other configurations of the described embodiments of the disclosed systems and methods are part of the scope of this disclosure. It will be appreciated that various of the above-disclosed and other features and functions, or alternatives thereof, may be desirably combined into many other different systems or applications. Also, various alternatives, modifications, variations or improvements therein may be subsequently made by those skilled in the art which are also intended to be encompassed by the following claims.

We claim:

1. A system for implementing an automated live event related game, comprising:
a display device that is configured to display information regarding a game matrix including a plurality of selectable gaming squares; and
a gamification device that is configured to
receive a request from a user via an information exchange device to participate in a gaming event as a player, the gaming event being associated with at least one live event;
receive additional entry information from the user specifically including a fantasy team or fantasy player selection;
receive an input from the user for application as a tie-breaker at the completion of at least one specified interval in the at least one live event;
assign one or more selectable gaming squares in the game matrix to each of a plurality of players;
direct display information identifying numbers associated with each of the assigned gaming squares to the each of the plurality of players on the display device;
monitor the progress of the at least one live event with which the game matrix is associated;
automatically obtain numbers indicative of the progress of the at least one live event at a plurality of intervals during the at least one live event;
automatically assign a point value to participation of each of the plurality of players based on a comparison of the obtained numbers from progress of the at least one live event and the identifying numbers associated with assigned gaming squares for the each of the plurality of players;
determine prospective winners from among the plurality of players based on a highest accrued point value at the plurality of intervals during the at least one live event and the identifying numbers associated with a particular selected gaming square;
apply additional winner verification criteria to the prospective winners to determine a final interval winner; and
provide notification to each final interval winner and the overall winner by sending information to the display device.

2. The system of claim 1, the additional winner verification criteria including applying a reactive scheme in which each of the prospective winners is (1) presented with advertising content, (2) presented with a series of queries regarding the advertising content, and (3) required to provide responses to the series of queries that will be evaluated by the gamification device to determine the final interval winner.

3. The system of claim 1, the additional winner verification criteria including an additional point value based on performance of the fantasy team or fantasy player selected by the user i that will be applied by the gamification device to determine the final interval winner.

4. The system of claim 1, the gamification device being further configured to aggregate the final interval winners and to apply the input from each of the final interval winners as to the outcome of the at least one live event at the completion of the at least one live event as the tie-breaker between the final interval winners to determine an overall winner.

5. The system of claim 1, the gamification device being further configured to
receive, from a user-controlled electronic device, user identification information; and
store the received user identification information in a storage device associated with the system.

6. The system of claim 1, the gamification device being further configured to
re-assign one or more selectable gaming squares in the game matrix to the each of the plurality of players at the completion of one or more of the plurality of intervals during the conduct of the live event; and
direct display information identifying numbers associated with each of the reassigned gaming squares to the each of the plurality of players on the display device.

7. The system of claim 1, the gamification device being further configured to assign the one or more selectable gaming squares in the game matrix to the each of the plurality of players based on at least one qualification criteria for the each of the plurality of players.

8. The system of claim 1, the gamification device being further configured to
aggregate assigned point values for the each of the plurality of players across a plurality of live events; and
provide notification to the plurality of players as to an aggregated points standing for the plurality of players.

9. The system of claim 1, the gamification device being further configured to
receive, from a user-controlled electronic device, an indication of at least one of a number of intervals in a single live event and a number of separate live events in which a particular player among the plurality of players chooses to participate;
track the particular player's accumulating aggregate point value across the indicated number of intervals in the single live event and the number of separate live events in which the particular player chooses to participate; and
provide notification to the particular player as to the aggregate point value accumulated across the indicated number of intervals in the single live event and the number of separate live events in which the particular player chooses to participate by sending information to the user-controlled electronic device of the particular player.

10. A non-transitory computer readable medium containing program instructions for causing a computer to perform a method comprising:
receiving a request from a user via an information exchange device to participate in a gaming event as a player, the gaming event being associated with at least one live event;
receiving additional entry information from the user specifically including a fantasy team or fantasy player selection;
receiving an input from the user for application as a tie-breaker at the completion of at least one specified interval in the at least one live event;
assigning one or more selectable gaming squares in the game matrix to each of a plurality of players;
directing display information identifying numbers associated with each of the assigned gaming squares to the each of the plurality of players on a display device;
monitoring the progress of the at least one live event with which the game matrix is associated;
obtaining numbers indicative of the progress of the at least one live event at a plurality of intervals during the at least one live event;
assigning a point value to participation of each of the plurality of players based on a comparison of the obtained numbers from progress of the at least one live event and the identifying numbers associated with assigned gaming squares for the each of the plurality of players;
determining prospective winners from among the plurality of players based on a highest accrued point value at the plurality of intervals during the at least one live event and the identifying numbers associated with a particular selected gaming square;
applying additional winner verification criteria to the prospective winners to determine a final interval winner; and
providing notification to each final interval winner and the overall winner by sending information to the display device.

11. The non-transitory computer readable medium 10, the additional winner verification criteria including applying a reactive scheme in which each of the prospective winners is (1) presented with advertising content, (2) presented with a series of queries regarding the advertising content, and (3) required to provide responses to the series of queries that will be evaluated by the gamification device to determine the final interval winner.

12. The non-transitory computer readable medium 10, the additional winner verification criteria including an additional point value based on performance of the fantasy team or fantasy player selected by the user i that will be applied by the gamification device to determine the final interval winner.

13. The non-transitory computer readable medium 10, the method further comprising:
aggregating the final interval winners; and
applying the input from each of the final interval winners as to the outcome of the at least one live event at the completion of the at least one live event as the tie-breaker between the final interval winners to determine an overall winner.

14. The non-transitory computer readable medium 10, the method further comprising:
receiving, from a user-controlled electronic device, user identification information; and
storing the received user identification information in a storage device associated with the system.

15. The non-transitory computer readable medium 10, the method further comprising:
re-assigning one or more selectable gaming squares in the game matrix to the each of the plurality of players at the completion of one or more of the plurality of intervals during the conduct of the live event; and
directing display information identifying numbers associated with each of the reassigned gaming squares to the each of the plurality of players on the display device.

16. The non-transitory computer readable medium 10, the method further comprising:
assigning the one or more selectable gaming squares in the game matrix to the each of the plurality of players based on at least one qualification criteria for the each of the plurality of players.

17. The non-transitory computer readable medium 10, the method further comprising:
aggregating assigned point values for the each of the plurality of players across a plurality of live events; and
providing notification to the plurality of players as to an aggregated points standing for the plurality of players.

18. The non-transitory computer readable medium 10, the method further comprising:
receiving, from a user-controlled electronic device, an indication of at least one of a number of intervals in a single live event and a number of separate live events in which a particular player among the plurality of players chooses to participate;
tracking the particular player's accumulating aggregate point value across the indicated number of intervals in the single live event and the number of separate live events in which the particular player chooses to participate; and
providing notification to the particular player as to the aggregate point value accumulated across the indicated number of intervals in the single live event and the number of separate live events in which the particular player chooses to participate by sending information to the user-controlled electronic device of the particular player.

19. A method for implementing an automated live event related game, comprising:
receiving a request from a user via an information exchange device to participate in a gaming event as a player, the gaming event being associated with at least one live event;
receiving additional entry information from the user specifically including a fantasy team or fantasy player selection;
receiving an input from the user for application as a tie-breaker at the completion of at least one specified interval in the at least one live event;
assigning one or more selectable gaming squares in the game matrix to each of a plurality of players;
directing display information identifying numbers associated with each of the assigned gaming squares to the each of the plurality of players on a display device;
monitoring the progress of the at least one live event with which the game matrix is associated;
obtaining numbers indicative of the progress of the at least one live event at a plurality of intervals during the at least one live event;
assigning a point value to participation of each of the plurality of players based on a comparison of the obtained numbers from progress of the at least one live event and the identifying numbers associated with assigned gaming squares for the each of the plurality of players;
determining prospective winners from among the plurality of players based on a highest accrued point value at the plurality of intervals during the at least one live event and the identifying numbers associated with a particular selected gaming square;
applying additional winner verification criteria to the prospective winners to determine a final interval winner; and
providing notification to each final interval winner and the overall winner by sending information to the display device.

20. The method of claim 19, the additional winner verification criteria including applying a reactive scheme in which each of the prospective winners is (1) presented with advertising content, (2) presented with a series of queries regarding the advertising content, and (3) required to provide responses to the series of queries that will be evaluated by the gamification device to determine the final interval winner.

21. The method of claim 19, the additional winner verification criteria including an additional point value based on performance of the fantasy team or fantasy player selected by the user i that will be applied by the gamification device to determine the final interval winner.

22. The method of claim 19, further comprising:
aggregating the final interval winners; and
applying the input from each of the final interval winners as to the outcome of the at least one live event at the completion of the at least one live event as the tie-breaker between the final interval winners to determine an overall winner.

23. The method of claim 19, further comprising:
receiving, from a user-controlled electronic device, user identification information; and
storing the received user identification information in a storage device associated with the system.

24. The method of claim 19, further comprising:
re-assigning one or more selectable gaming squares in the game matrix to the each of the plurality of players at the completion of one or more of the plurality of intervals during the conduct of the live event; and
directing display information identifying numbers associated with each of the reassigned gaming squares to the each of the plurality of players on the display device.

25. The method of claim 19, further comprising:
assigning the one or more selectable gaming squares in the game matrix to the each of the plurality of players based on at least one qualification criteria for the each of the plurality of players.

26. The method of claim 19, further comprising:
aggregating assigned point values for the each of the plurality of players across a plurality of live events; and
providing notification to the plurality of players as to an aggregated points standing for the plurality of players.

27. The method of claim 19, further comprising:
receiving, from a user-controlled electronic device, an indication of at least one of a number of intervals in a single live event and a number of separate live events in which a particular player among the plurality of players chooses to participate;
tracking the particular player's accumulating aggregate point value across the indicated number of intervals in the single live event and the number of separate live events in which the particular player chooses to participate; and
providing notification to the particular player as to the aggregate point value accumulated across the indicated number of intervals in the single live event and the number of separate live events in which the particular player chooses to participate by sending information to the user-controlled electronic device of the particular player.
